(19) United States
(12) Patent Application Publication
(10) Pub. No.: US 2003/0235868 A1
(43) Pub. Date: Dec. 25, 2003

(54) Antibodies specific for mucin polypeptide

(75) Inventors: Hendricus R.J.M. Hoogenboom, Maastricht (NL); Maria Paulina Godelieve, Hasselt (BE); Albert S.B. Edge, Newton, MA (US)

Correspondence Address: PALMER & DODGE, LLP, KATHLEEN M. WILLIAMS, 111 HUNTINGTON AVENUE, BOSTON, MA, 02199, US

(73) Assignee: DYAX Corporation

(21) Appl. No.: 10/417,312

(22) Filed: Apr. 16, 2003

Related U.S. Application Data
(60) Provisional application No. 60/374,432, filed on Apr. 22, 2002.

(51) Int. Cl. G01N 33/53; G01N 33/567; C07K 16/18; C07K 7/64

(52) U.S. Cl. 435/7.2; 530/388.25; 530/317

(57) ABSTRACT

Antibodies and peptide ligands are described herein, which are specific for epitopes on MUC-H, which reside on the MUC1 extracellular fragment remaining on the cell surface after cleavage of the MUC1 protein.

### RELATED APPLICATION

This application claims the benefit of U.S. Provisional Application No. 60/374,432, filed on Apr. 22, 2002. The entire teachings of that application are incorporated herein by reference.

### BACKGROUND

MUC1 is a transmembranous glycoprotein that is expressed on the apical surface of many epithelial cells, e.g., breast, ovary, bladder, lung (Zotter, S. et al., (1988) Cancer Rev. 11-12:56-101; Winterford, C. M. et al. (1999) J. Histochem. Cytochem. 47:1063-74) with a molecular weight of more than 400 kD. The MUC1 gene is located on chromosome 1q21-24 (Swallow, D. M. et al. (1987) Nature 328:82-4) and contains seven exons (Lancaster, C. A. et al. (1990) Biochem. Biophys. Res. Commun. 173:1019-29). The MUC1 protein consists of three parts, a large extracellular domain, a transmembrane domain (Ligtenberg, M. J. et al. (1992) J. Biol. Chem. 267:6171-7), and a cytoplasmic tail. It can exist in two forms, a membrane glycoprotein, or a shed form. Upon conversion to the shed form of the protein, the extracellular domain is cleaved off near the cell surface, possibly by kallikrein-like serine protease, leaving behind a short “stubble” protein on the extracellular surface of the membrane. Two predicted cleavage sites for the extracellular domain (to release the domain containing the VNTR (Variable Number Tandem Repeat region) from the membrane bound domain) are 65 and possibly also 52 amino acids upstream of the transmembrane domain as determined by Parry et al. (Parry, S. et al. (2001) Biochem. Biophys. Res. Commun. 283:715-20). The proteolytic sites are respectively between the sequences FRPG and SVVV, and between the sequences FREG and TINV. It is thought that after this clipping, there is an association between the transmembranous and extracellular part, but the nature of this association remains unknown (Parry, S. et al. (2001) Biochem. Biophys. Res. Commun. 283:715-20). The shed extracellular MUC1 is the N-terminal part behind the cleavage site (Wreschner, D. H. et al. (1990) Eur. J. Biochem. 189:463-73; Ligtenberg, M. J. et al. (1992) J. Biol. Chem. 267:6171-6177). The extracellular part contains a variable number tandem repeat (VNTR) region, and the number repeats differs from individual to individual, and can vary in the Northern European population between 21 and 125 repeats (Gendler, S. J. (1990) J. Biol. Chem. 265:15286-15293).

The extracellular part has a rod-like, rigid conformation due to the folding of the repeat region, and extends 200 to 500 nm above the plasma membrane (Hilkens, J. (1992) Trends Biochem. Sci. 17:359-363). The MUC1 tandem repeat is highly glycosylated because every repeat has five potential O-glycosylation sites, and the carbohydrate content of MUC1 can be more than 50% (Gendler, S. J. (1990) J. Biol. Chem. 265:15286-15293; Muller, S. et al. (1997) J. Biol. Chem. 272:24780-93; Patton, S. et al. (1995) Biochim. Biophys. Acta 1241:407-23). In tumor cells, e.g., epithelial adenocarcinoma, MUC1 is overexpressed and differentially glycosylated as compared to normal tissues, exposing new cryptic peptide and carbohydrate epitopes. In normal tissues, MUC1 is expressed at the apical site of the cell, while in tumor tissues MUC1 is expressed in the entire cell. Upon cleavage to the shed form, the extracellular N-terminal part of MUC1, containing the tandem repeat, is shed, leaving the transmembranous C-terminal part on the cell surface. Via a putative interaction between this shed form of MUC1 and the remaining extracellular region of MUC1 linked to a transmembranous and intracellular region (MUC1-Stubble), the shed material may remain for some time on the cell surface, but eventually, via an unknown mechanism, will be shed.

The MUC1 protein also contains a SEA domain, (“Sea urchin sperm protein—Enterokinase—Agrin” domain). In this and other proteins this domain is suggested as regulating or binding carbohydrate structures (Bork, P. et al. (1995) Protein Sci. 4:1421-5). In MUC1, this domain follows the tandem repeat region, and is followed by the transmembrane region. It has been hypothesized that it is the SEA domain that interacts with glycosylated residues on the large extracellular fragment and thus serves to retain the shed form of MUC-1 on the cell surface. Within the SEA domain the putative protease cleavage site is still present; it is unknown whether it is the intact SEA domain (in non-clipped MUC1) or alternatively the clipped C-terminal part of the SEA domain (as part of the MUC-Stubble) which could be responsible for the interaction with the shed form of MUC1.

A number of splice variants of the MUC1 protein have been isolated. These are splice variants devoid of the tandem repeats, and therefore consist of a short N-terminal region, most of the SEA domain, the transmembrane region and the intracellular region. These include MUC1/X, MUC1/X/alt, MUC1/Y, MUC1/Y/alt, MUC1/V and MUC1/V/alt, and antibodies have been made to these proteins (Wreschner, WO 96/03502). These proteins are missing the tandem repeat array normally found in the full-length protein. MUC1/Y is tumor-associated (Baruch, A. et al. (1997) Int. J. Cancer 71:741-9). MUC1/Y is not cleaved at the enzymatic cleavage sites as described for MUC1. Hartman et al ((1999), Int. J. Cancer 82:256-267) also teaches monoclonal antibodies to the MUC1/Y protein, including one that reacts with the 30 N-terminal amino acids of the extracellular domain of MUC1, and another that reacts with the 14 C-terminal amino acids of the extracellular domain of MUC1. However these antibodies are not specific for either MUC1/Y or for the extracellular domain remaining on the cell after protease cleavage since they would also recognize MUC1.

Many antibodies to the tumor-associated peptide epitopes of the tandem repeat bind relatively specifically to epithelial tumors. Those antibodies often also bind to serum MUC1 but not always to the same extent (Norum, L. F. et al. (1998) Tumour Biol. 19(Suppl 1): 134-46). Antibodies against the tandem repeat have different internalization properties (Pietersz, G. A. (1997) Cancer Immunol. Immunother. 44:323-8). Antibodies and peptides against the tandem repeat of MUC1 are successfully used in radio immune scintigraphy and radio immunotherapy. In such therapies, the efficiency of some anti-MUC1 antibodies is hampered by binding of the antibody to shed MUC1 in the serum of the patient, which can cause problems when antibodies are injected intravenously. For example the dosage of the antibody may have to be such that most of the antibody will be binding to the serum MUC1, leaving little available for binding to cellular MUC1. Further the formation of large immune complexes between shed MUC1, with multiple epitopes, and antibodies with one or more binding sites, may lead to and to accumulation of the complexes in the liver, rapid clearance and toxic side effects associated with this altered pharmacokinetics (e.g. serum sickness). If the antibody is labeled with cytotoxic drugs or radio isotopes, such effects may be detrimental to the antibody's efficacy.

Therefore antibodies that would not recognize the shed form of MUC1 but do recognize the remaining part of MUC1 on the cell surface, would not suffer these therapeutic drawbacks, and could be superior targeting agents.

Another disadvantage of many anti-MUC1 antibodies is that they target epitopes that are relatively far removed from the cell surface, reducing the efficacy of natural antibody-mediated immune effectors such as Complement-Dependent Cytotoxicity. Therefore targeting of tumor cells with antibodies to MUC1 epitopes that are more closely located to the cell surface may dramatically increase their anti-tumor cell efficacy.

### SUMMARY OF THE INVENTION

This invention provides, in part, polypeptide ligands that specifically bind to MUC1 related epitopes that we have grouped together as MUC1-H. Antibodies to MUC1-H recognize epitopes that are associated with the MUC1-stubble or the complex formed between soluble MUC1 and the MUC1-Stubble complex, and are thus devoid of binding with equivalent affinity to shed MUC1. MUC1-H comprises epitopes that are not present on shed MUC1 but are present on any cell-surface expressed form of MUC1. MUC1-H also comprises epitopes that are hidden in full length, non-clipped MUC1 (or non-clipped splice variants of MUC1), and only appear on the cell-surface bound stump of MUC1 after specific cleavage of the MUC1 (or splice variants of MUC1), hence the name MUC1-H(idden). Further, MUC1-H contains those epitopes that are formed between the shed form of MUC1 that remains associated with the MUC-stubble (possibly derived from an alternative splicing form of MUC1) and the MUC-stubble itself; such conformational epitopes are likely to be present only on cell-surface associated MUC1 and not on serum MUC1. Most of the hidden epitopes on MUC1-H will be based within the C-terminal amino acids of the extracellular region of MUC1 (the MUC-Stubble), and can be linear or conformational epitopes within this sequence that are normally not detectable by a given polypeptide ligand on non-clipped MUC1 or splice variant of MUC1. The MUC-Stubble may consist of 65 or 52 amino acids upstream of the transmembrane region of MUC1 as defined by Parry et al., but could also start at other N-terminal residues in this region depending on the site-specificity of the proteases cleaving the protein. For example, such epitopes can comprise the region on the MUC-Stubble that is responsible for the interaction with the shed form of MUC1 (such as the region encoded by the SEA domain). Assuming such epitopes are normally covered due to interaction with shed MUC1, upon release of the latter they become exposed. Other MUC1-H epitopes comprise conformational epitopes formed by the complex of the N-terminal region of MUC1 after clipping at the junction with the Stubble, and the stubble itself; in such case the sequence will contain at least part of the C-terminal 65 amino acids of the extracellular region of MUC1. MUC1-H epitopes may be formed by a linear or conformational sequence within the representative sequence of the MUC1-Stubble as is set out in SEQ ID NO: 2. “Binding of a ligand to SEQ ID NO: 2” refers not only to the interaction of the ligand with any epitope composed of the full sequence of SEQ ID NO: 2, but also epitopes composed of those fragments of SEQ ID NO: 2 that are shorter due to cleavage at alternative cleavage sites at the N-terminus. For example, this would include epitopes composed of the 52 amino acid sequence starting at the 14th residue of the SEQ ID NO: 2. In a preferred embodiment, the polypeptide ligands are antibodies (the term of which includes antigen-binding fragments). In another preferred embodiment, the polypeptide ligands are modified scaffold polypeptides or peptides. In still another preferred embodiment, the peptides (e.g., polypeptides of 6-25 amino acids) are cyclic peptides or linear peptides. A polypeptide ligand may be a multi-chain protein (e.g., including at least two peptides or polypeptides). An example of a multi-chain protein is an antibody. Whereas many examples described herein refer to antibody ligands, it is understood, that the invention can be practiced using any polypeptide ligand (e.g., antibody and non-antibody ligand) provided herein.

The invention features an isolated polypeptide ligand which specifically binds to an epitope on MUC1 that is not present on shed MUC1 but is present on any cell-surface expressed form of MUC1. The isolated polypeptide ligand can be such that the polypeptide ligand does not bind the VNTR region of the MUC1 protein. The polypeptide ligand can be a peptide ligand, e.g., a scaffold peptide, a linear peptide, or a cyclic peptide. The polypeptide ligand can be an antibody. The antibody can specifically binds a polypeptide consisting of SEQ ID NO: 2 or fragments thereof (e.g., SEQ ID NO: 3), or the antibody can be an antibody which competes with such an antibody (e.g., competes for binding to SEQ ID NO: 2 or fragments thereof (e.g., SEQ ID NO: 3), or competes for binding to a complex between SEQ ID NO: 2 and SEQ ID NO: 1, or competes for binding to a complex between SEQ ID NO: 3 and SEQ ID NO: 1). The antibody can specifically bind a polypeptide consisting of the N-terminal 51 amino acids of SEQ ID NO: 2, or can specifically bind a polypeptide consisting of the N-terminal 38 amino acids of SEQ ID NO: 2. The antibody can be a human antibody or an intact immunoglobulin. The antibody can be conjugated to a functional moiety, e.g., a drug, a cytotoxic agent, a detectable moiety, or a solid support.

The invention also features an isolated polypeptide ligand which binds to an epitope on MUC1 that is not present on shed MUC1 and is not present on the MUC1-Stubble when part of the full-length non-clipped MUC1 protein. The isolated polypeptide ligand can be such that the polypeptide ligand does not bind the VNTR region of the MUC1 protein. The polypeptide ligand can be a peptide ligand, e.g., a scaffold peptide, a linear peptide, or a cyclic peptide. The polypeptide ligand can be an antibody. The antibody can specifically binds a polypeptide consisting of SEQ ID NO: 2 or fragments thereof (e.g., SEQ ID NO: 3), or the antibody can be an antibody which competes with such an antibody (e.g., competes for binding to SEQ ID NO: 2 or fragments thereof (e.g., SEQ ID NO: 3), or competes for binding to a complex between SEQ ID NO: 2 and SEQ ID NO: 1, or competes for binding to a complex between SEQ ID NO: 3 and SEQ ID NO: 1). The antibody can specifically bind a polypeptide consisting of the N-terminal 51 amino acids of SEQ ID NO: 2, or can specifically bind a polypeptide consisting of the N-terminal 38 amino acids of SEQ ID NO: 2. The antibody can be a human antibody or an intact immunoglobulin. The antibody can be conjugated to a functional moiety, e.g., a drug, a cytotoxic agent, a detectable moiety, or a solid support.

The invention additionally features an isolated polypeptide ligand which specifically binds the C-terminal 65 amino acids of the extracellular region of the MUC1 protein. The isolated polypeptide ligand can be such that the polypeptide ligand does not bind the VNTR region of the MUC1 protein. The polypeptide ligand can be a peptide ligand, e.g., a scaffold peptide, a linear peptide, or a cyclic peptide. The polypeptide ligand can be an antibody. The antibody can specifically binds a polypeptide consisting of SEQ ID NO: 2 or fragments thereof (e.g., SEQ ID NO: 3), or the antibody can be an antibody which competes with such an antibody (e.g., competes for binding to SEQ ID NO: 2 or fragments thereof (e.g., SEQ ID NO: 3), or competes for binding to a complex between SEQ ID NO: 2 and SEQ ID NO: 1, or competes for binding to a complex between SEQ ID NO: 3 and SEQ ID NO: 1). The antibody can specifically bind a polypeptide consisting of the N-terminal 51 amino acids of SEQ ID NO: 2, or can specifically bind a polypeptide consisting of the N-terminal 38 amino acids of SEQ ID NO: 2. The antibody can be a human antibody or an intact immunoglobulin. The antibody can be conjugated to a functional moiety, e.g., a drug, a cytotoxic agent, a detectable moiety, or a solid support.

In an additional aspect, the invention features an isolated polypeptide ligand which specifically binds the C-terminal 52 amino acids of the extracellular region of the MUC1 protein. The isolated polypeptide ligand can be such that the polypeptide ligand does not bind the VNTR region of the MUC1 protein. The polypeptide ligand can be a peptide ligand, e.g., a scaffold peptide, a linear peptide, or a cyclic peptide. The polypeptide ligand can be an antibody. The antibody can specifically binds a polypeptide consisting of SEQ ID NO: 2 or fragments thereof (e.g., SEQ ID NO: 3), or the antibody can be an antibody which competes with such an antibody (e.g., competes for binding to SEQ ID NO: 2 or fragments thereof (e.g., SEQ ID NO: 3), or competes for binding to a complex between SEQ ID NO: 2 and SEQ ID NO: 1, or competes for binding to a complex between SEQ ID NO: 3 and SEQ ID NO: 1). The antibody can specifically bind a polypeptide consisting of the N-terminal 51 amino acids of SEQ ID NO: 2, or can specifically bind a polypeptide consisting of the N-terminal 38 amino acids of SEQ ID NO: 2. The antibody can be a human antibody or an intact immunoglobulin. The antibody can be conjugated to a functional moiety, e.g., a drug, a cytotoxic agent, a detectable moiety, or a solid support.

In a further aspect, the invention features an isolated non-antibody ligand which specifically binds a polypeptide consisting of SEQ ID NO: 2 or fragments thereof. The isolated polypeptide ligand can be such that the polypeptide ligand does not bind the VNTR region of the MUC1 protein. The polypeptide ligand can be a peptide ligand, e.g., a scaffold peptide, a linear peptide, or a cyclic peptide. The polypeptide ligand can be an antibody. The antibody can specifically binds a polypeptide consisting of SEQ ID NO: 2 or fragments thereof (e.g., SEQ ID NO: 3), or the antibody can be an antibody which competes with such an antibody (e.g., competes for binding to SEQ ID NO: 2 or fragments thereof (e.g., SEQ ID NO: 3), or competes for binding to a complex between SEQ ID NO: 2 and SEQ ID NO: 1, or competes for binding to a complex between SEQ ID NO: 3 and SEQ ID NO: 1). The antibody can specifically bind a polypeptide consisting of the N-terminal 51 amino acids of SEQ ID NO: 2, or can specifically bind a polypeptide consisting of the N-terminal 38 amino acids of SEQ ID NO: 2. The antibody can be a human antibody or an intact immunoglobulin. The antibody can be conjugated to a functional moiety, e.g., a drug, a cytotoxic agent, a detectable moiety, or a solid support.

In another aspect, the invention features a method of detecting MUC1-H in a sample, where the method includes: (a) providing a sample; (b) contacting the sample of (a) with a polypeptide ligand which specifically binds a polypeptide comprising MUC1-H under conditions which permit binding of the polypeptide ligand to MUC1-H; and (c) detecting binding of the polypeptide ligand with MUC1-H in the sample, where detection of binding indicates the presence of MUC1-H in the sample; thereby detecting MUC1-H in the sample.

In another aspect, the invention features a method of identifying a polypeptide ligand specific for MUC1-H, where the method includes: (a) providing a phage library comprising phage expressing candidate MUC1-H binding polypeptides; (b) contacting said phage library with MUC1-H protein; and (c) detecting binding of the MUC1-H protein to phage; thereby identifying a polypeptide ligand specific for MUC1-H.

In an additional aspect, the invention features a method of killing a cell, where the method includes: (a) providing a cell; (b) contacting the cell of (a) with a polypeptide ligand which specifically binds a polypeptide comprising MUC1-H under conditions which permit binding of the polypeptide ligand to MUC1-H; thereby killing the cell.

By “specifically binds” is meant that the polypeptide ligand does not include prior art antibodies which are anti-cytoplasmic domain antibodies, or antibodies which bind MUC/Z, MUC/X, Muc1/X/alt, MUC1/Y, MUC1/Y/alt, MUC1/V, MUC1/V/alt, MUC1/W, MUC1/W/alt, MUC1/Z, or MUC1/Z/alt, as described in Wreshner, WO 96/03502, or the 10D2/36 antibody described in Hartman et al., 1999, Int. J. Cancer 82:256-267) the M29 and 232A antibodies described by Hilkens et al. (1998) Tumor Biol. 19(suppl 1):67-70), of which hereby incorporated by reference. The antibodies to MUC1-H do not recognize the structures recognized by these known antibodies. These known antibodies bind to epitopes that are present in the intact MUC1 or the full length MUC1/Y as well as being within the stubble sequence. The antibodies of this invention do not recognize the epitopes defined by these antibodies.

The Wreschner and Hartman et al. prior art antibodies bind at least one of the following sequences: MUC1 protein (SEQ ID NO: 1), MUC1/X protein (SEQ ID NO: 3), MUC1/X/alt protein (SEQ ID NO: 4), MUC1/Y protein (SEQ ID NO: 5), MUC1/Y/alt protein (SEQ ID NO: 6), MUC1/V protein (SEQ ID NO: 7), MUC1/V/alt protein (SEQ ID NO: 8).

In a preferred embodiment, the polypeptide ligand does not bind the VNTR region of the MUC1 protein or shed MUC1 protein.

The invention features a purified polypeptide ligand which specifically binds MUC1-H epitopes within the C-terminal 65 amino acids or the C-terminal 52 amino acids of the extracellular region of the MUC1 protein. Preferably, this ligand does not bind the VNTR region of the MUC1 protein. This ligand recognizes the epitope in this sequence only after the release of the VNTR region by a protease.

The invention also features a purified antibody which specifically binds a polypeptide consisting of SEQ ID NO: 2, and also a purified second antibody which can compete with such an antibody for binding to SEQ ID NO: 2. The invention also features a purified antibody which specifically binds a polypeptide consisting of a conformational epitope revealed by the cleavage of the shed form of full-length MUC1, but does not bind to a polypeptide consisting of the shed form if MUC1. The invention also features a purified antibody which specifically binds a polypeptide consisting of a conformational epitope with contributions from SEQ ID NO: 1 (the MUC1 sequence) and SEQ ID NO: 2 but does not bind a polypeptide consisting of SEQ ID NO: 1. The invention also features a purified second antibody which can compete with above antibodies for binding to SEQ ID NO: 2. “Compete with”, in the context of an antibody which can compete with another antibody means that in a cell binding flow cytometric analysis or in a specific test (e.g., ELISA, RIA, etc.), it can reduce the binding of a MUC1-H specific antibody with at least 50% when used at a 10 to 100-fold molar excess.

The invention also features a purified antibody which specifically binds a polypeptide consisting of the first 51 amino acids of SEQ ID NO: 2, and also a purified antibody that specifically binds to an epitope of a polypeptide consisting of the first 51 amino acids of SEQ ID NO: 2. The invention also features a purified antibody which specifically binds a polypeptide consisting of a conformational epitope with contributions from SEQ ID NO: 1 and SEQ ID NO: 2 but does not bind a polypeptide consisting of SEQ ID NO: 1, and also a purified second antibody which can compete with such an antibody for binding to the first 51 amino acids of SEQ ID NO: 2.

The invention also features an isolated non-antibody peptide which specifically binds a polypeptide consisting of SEQ ID NO: 2,and preferably which does not bind the VNTR region of the MUC1 protein. The invention also features an isolated non-antibody peptide which specifically binds a polypeptide consisting of SEQ ID NO: 2, and also a purified second non-antibody peptide which can compete with such a peptide for binding to SEQ ID NO: 2. The invention also features a purified non-antibody peptide which specifically binds a polypeptide consisting of a comformational epitope revealed by the cleavage of the shed form of full-length MUC1, but does not bind to a polypeptide consisting of the shed form if MUC1. The invention also features a purified non-antibody peptide which specifically binds a polypeptide consisting of a conformational epitope with contributions from SEQ ID NO: 1 (the MUC1 sequence) and SEQ ID NO: 2 but does not bind a polypeptide consisting of SEQ ID NO: 1. The invention also features a purified second non-antibody peptide which can compete with above antibodies and peptides for binding to SEQ ID NO: 2.

The invention also features a purified polypeptide which specifically binds the C-terminal region of the MUC1 protein, and wherein said polypeptide does not bind the VNTR region of the MUC1 protein. The invention also features a purified polypeptide that specifically binds the MUC1-H protein, wherein said polypeptide does not bind the VNTR region of the MUC1 protein. The invention also features a purified polypeptide which specifically binds a polypeptide consisting of the first 51 or 38 amino acids of SEQ ID NO: 2. The invention also features a purified polypeptide which specifically binds a polypeptide consisting of a conformational epitope with contributions from SEQ ID NO: 1 (the MUC1 sequence) and SEQ ID NO: 2 but does not bind a polypeptide consisting of SEQ ID NO: 1, and wherein said polypeptide does not bind the VNTR region of the MUC1 protein. The invention also features a purified polypeptide that specifically binds to an epitope of a polypeptide consisting of the first 51 amino acids of SEQ ID NO: 2, and also a purified second polypeptide which can compete with such a polypeptide for binding to the first 51 or 38 amino acids of SEQ ID NO: 2.

In another aspect, the invention features a method of detecting MUC1-H in a sample, the method comprising: (a) providing a sample; (b) contacting the sample of (a) with a polypeptide ligand which specifically binds a polypeptide comprising SEQ ID NO: 2 under conditions which permit binding of the polypeptide ligand to MUC1-H; and (c) detecting binding of the polypeptide ligand with MUC1-H in the sample, wherein detection of binding indicates the presence of MUC1-H in the sample; thereby detecting MUC1-H in the sample. The MUC1-H detected in the sample may be free, or may be attached to a biological entity, e.g., a cell or a phage, where it can be displayed as an epitope.

The invention also features a method of identifying a polypeptide ligand specific for MUC1-H, comprising (a) providing a phage library comprising phage expressing candidate MUC1-H binding polypeptides; (b) contacting said phage library with MUC1-H protein; and (c) detecting binding of the MUC1-H protein to phage; thereby identifying a polypeptide ligand specific for MUC1-H. The MUC1-H detected in the sample may be free, or may be attached to a biological entity, e.g., a cell or a phage, where it can be displayed as an epitope.

The invention also features a method of ablating or killing a MUC1- or MUC1-H-bearing cell by contacting the cell with a polypeptide ligand according to the invention under conditions which permit cell ablation. Preferably, this method is performed in vitro or ex vivo.

The polypeptide ligands of the present invention can be a peptide ligand, e.g., a scaffold peptide, a linear peptide, or a cyclic peptide. The polypeptide ligand can be an antibody. The antibody can be a human antibody, a chimeric antibody, a recombinant antibody, a humanized antibody, a monoclonal antibody, or a polyclonal antibody. The antibody can be an intact immunoglobulin, e.g., an IgA, IgG, IgE, IgD, IgM or subtypes thereof. The antibody can be conjugated to a functional moiety (e.g., a compound which has a biological or chemical function( which may be a second different polypeptide, a therapeutic drug, a cytotoxic agent, a detectable moiety, or a solid support.

The polypeptide ligands of the present invention can be conjugated to a functional moiety, e.g., a second polypeptide, a drug, a cytotoxic agent, a detectable moiety, or a solid support.

By “MUC1 protein” is meant a full-length, polymorphic, high molecular weight glycoprotein that is normally expressed by secretory epithelial cells, but abundantly expressed in malignant breast epithelial cells, and when expressed by non-malignant cells tends to be differentially glycosylated with O-glycosidic-linked carbodydrate side chains, taking on normal mucin-like characteristics. The MUC1 gene product is a transmembrane protein that contains a large extracellular domain, a transmembrane domain and a cytoplasmic tail. The extracellular domain contains a variable number tandem repeat (VNTR) region with a 20 amino acid repeat motif that varies in number from about 20 to about 100 repeats. One such MUC1 protein is presented in SEQ ID NO: 1.

By “MUC1-H” protein is meant the collection of epitopes found on the MUC1 polypeptide which are present within the MUC1-protein or derivatives of the protein residing on the cell surface after cleavage of the MUC1, and are not present on shed MUC1 nor on full-length non-clipped MUC1, and are different from and. Epitopes can be based within or are partially formed by the MUC-1 Stubble. Epitopes may be exposed only after the cleavage has occurred, or after the clipped N-terminal region of MUC1 is disassociated from the cell surface. Representative epitopes corresponding to MUC1-H will be present in SEQ ID NO: 2 or in the complex formed by SEQ ID NO: 2 and the clipped form of MUC1 or in the complex formed by SEQ ID NO: 2 and SEQ ID NO: 1.

By “MUC1/X” is meant a splice variant of the MUC1 protein, as shown in SEQ ID NO: 3. Likewise, “MUC1/X/alt” (SEQ ID NO: 4), “MUC1/Y” (SEQ ID NO: 5), “MUC1/Y/alt” (SEQ ID NO: 6), “MUC1/V” (SEQ ID NO: 7) and “MUC1/V/alt” (SEQ ID NO: 8) are also splice variants of the MUC1 protein.

By “VNTR region” or the equivalent term “tandem repeat array” is meant the region of the full-length MUC1 protein which consists of variable number of tandem repeats. The repeat unit is generally the 20 amino acid tandem repeat unit PDTRPAPGSTAPPAHGVTSA, with some variations possible between repeats. The precise number of repeats can vary from individual to individual, and can vary in the Northern European population between 21 and 125 repeats (Gendler, S. J. (1990) J. Biol. Chem. 265:15286-15293).

An “epitope” or “antigenic determinant”, as used herein, refers to that portion of a molecule that makes contact with a particular polypeptide ligand binding the MUC1-H. When a protein or fragment of a protein is used to immunize a host animal, numerous regions of the protein may induce the production of antibodies which bind specifically to a given region or three-dimensional structure on the protein; these regions or structures are referred to as epitopes or antigenic determinants. An epitope or antigenic determinant may compete with the intact antigen (i.e., the immunogen used to elicit the immune response) for binding to an antibody. Epitopes can be linear, comprising essentially a linear sequence from the antigen, or conformational, comprising sequences which are genetically separated by other sequences but come together structurally at the binding site for the polypeptide ligand.

The anti-MUC1-H ligands bind to human MUC1-H with high affinity and specificity, and thus can be used as diagnostic, prophylactic, or therapeutic agents in vivo and in vitro. Preferably the ligands specifically bind to the MUC1-H. As used herein, “specific binding” refers to the property of the antibody: (1) to bind to MUC1-H, e.g., human MUC1-H, with an affinity of at least 1×10⁷ M⁻¹, and (2) to preferentially bind to MUC1-H, e.g., human MUC1-H, with an affinity that is at least two-fold, 50-fold, 100-fold, or greater than its affinity for binding to a non-specific antigen (e.g., BSA, casein) other than MUC1-H.

Accordingly, the invention provides anti-MUC1-H antibodies, antibody fragments, and pharmaceutical compositions thereof, as well as nucleic acids, recombinant expression vectors and host cells for making such antibodies and fragments. Methods of using the antibodies of the invention to detect MUC1-H, or to ablate or kill a MUC1-H-expressing cell, e.g., a MUC1-H-expressing cancer cell, either in vitro or in vivo, are also encompassed by the invention. By “ablate or kill” is meant that the cell is destroyed or rendered non-functional, that is, the cell can be completely destroyed, or rendered incapable of further division, or signaling the division of other cells.

The polypeptide ligands of the invention interact with, e.g., bind to MUC1-H, preferably human MUC1-H, with high affinity and specificity. For example, the polypeptide ligand binds to human MUC1-H with an affinity constant of at least 10⁷ M⁻¹, preferably, at least 10⁸ M⁻¹, 10⁹ M⁻¹, or 10¹⁰ M⁻¹. Preferably, the polypeptide ligand interacts with, e.g., binds to, the extracellular MUC1-H, or “stubble” portion of the MUC1 protein, and most preferably, the extracellular MUC1-H, or “stubble” portion of the human MUC1 protein (e.g., SEQ ID NO: 2).

In one embodiment, the anti-MUC1-H ligand binds all or part of the epitope of an antibody described herein. The anti-MUC1-H ligand can inhibit, e.g., competitively inhibit, the binding of an antibody described herein, to human MUC1-H. An anti-MUC1-H ligand may bind to an epitope, e.g., a conformational or a linear epitope, which epitope when bound prevents binding of an antibody described herein. The epitope can be in close proximity spatially or functionally-associated, e.g., an overlapping or adjacent epitope in linear sequence or conformationally to the one recognized by the antibody. In one embodiment, the anti-MUC1-H ligand binds to an epitope located wholly or partially within the region of about amino acid 253 to about amino acid 382 of human MUC1 (SEQ ID NO: 1). Preferably, the epitope includes at least part of the SEA domain of human MUC1, and/or the collection of epitopes found on the MUC1 polypeptide which are present within the MUC1-protein or derivatives of the protein residing on the cell surface after cleavage of the MUC1, and are not present on shed MUC1 nor on full-length non-clipped MUC1, and are different from the epitopes defined by antibodies 6D3/12, 10D2/36, 232A or M29, that recognize regions within the stubble. Such epitopes can be based within or are partially formed by the MUC-1 Stubble. Epitopes may be exposed only after the clevage has occurred, or after the clipped N-terminal region of MUC1 is disassociated from the cell surface. Representative epitopes corresponding to MUC1-H will be present in SEQ ID NO: 2 or in the complex formed by SEQ ID NO: 2 and SEQ ID NO: 1.

Epitopes comprising human MUC1-H are expressed on epithelial cells and on many tumor cells. The antibodies of the invention bind to the cell surface of these cells, and in particular, to the cell surface of the living cells. The distinct genetic and structural make up of tumor cells over normal epithelial cells may lead to a preferential exposure of MUC1-H epitopes on certain tumor cells or a fraction of tumor cells. Particularly in tumors this may be caused by a higher MUC1 expression level, an altered protease expression spectrum, altered quantity and quality of glycosylation, and/or by a modified proteolytic cleavage pattern and non-polar expression pattern. The presence of MUC1-H much closer to the cell surface when compared with the VNTR epitopes is expected to yield a stronger antibody Fc-mediated anti-tumor cell activity than seen with anti-VNTR antibodies. Preferably, the polypeptide ligands of the present invention are also internalized with the MUC1-H, which permits the intracellular delivery of an agent conjugated to the antibody, e.g., a cytotoxic or a labeling agent. Accordingly, the polypeptide ligands of the invention can be used to target living normal, benign hyperplastic, and cancerous cells that express the MUC1-H protein. Such internalization may be based on the natural internalization of the bound MUC1 protein variant, or may be enhanced or created by signals provide on the polypeptide ligand.

In a preferred embodiment, the polypeptide ligand is an antibody. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A. et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917, which are incorporated herein by reference in their entirety). Each VH and VL is composed of three CDR's and four FRs, arranged from amino-terminus to carboxy-terminus in the following order: FR1, CDR1, FR2, CDR2, FR3, CDR3, FR4.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are interconnected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The term “antibody” includes intact immunoglobulins of types IgA, IgG, IgE, IgD, IgM (as well as subtypes thereof), wherein the light chains of the immunoglobulin may be of types kappa or lambda.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

The term “antibody” also encompasses antigen-binding fragments of an antibody. The term “antigen-binding fragment” of an antibody (or simply “antibody portion,” or “fragment”), as used herein, refers to one or more fragments of a full-length antibody that retain the ability to specifically bind to MUC1-H (e.g., human MUC1-H). Examples of binding fragments encompassed within the term “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies.

The antibody is preferably monospecific, e.g., a monoclonal antibody, or antigen-binding fragment thereof. The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition.

The anti-MUC1-H antibodies can be full-length (e.g., an IgG (e.g., an IgG1, IgG2, IgG3, IgG4), IgM, IgA (e.g., IgA1, IgA2), IgD, and IgE, but preferably an IgG) or can include only an antigen-binding fragment (e.g., a Fab, F(ab′)₂ or scFv fragment). The antibody, or antigen-binding fragment thereof, can include two heavy chain immunoglobulins and two light chain immunoglobulins, or can be a single chain antibody. The antibodies can, optionally, include a constant region chosen from a kappa, lambda, alpha, gamma, delta, epsilon or a mu constant region gene. A preferred anti-MUC1-H antibody includes a heavy and light chain constant region substantially from a human antibody, e.g., a human IgG1 constant region or a portion thereof. As used herein, “isotype” refers to the antibody class (e.g., IgM or IgG1) that is encoded by heavy chain constant region genes.

In a preferred embodiment, the antibody (or fragment thereof) is a recombinant or modified anti-MUC1-H antibody, e.g., a chimeric, a humanized, a deimmunized, or an in vitro generated antibody. The term “recombinant” or “modified” human antibody, as used herein, is intended to include all antibodies that are prepared, expressed, created or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes or antibodies prepared, expressed, created or isolated by any other means that involves splicing of human immunoglobulin gene sequences to other DNA sequences. Such recombinant antibodies include humanized, CDR grafted, chimeric, deimmunized, in vitro generated antibodies, and may optionally include constant regions derived from human germline immunoglobulin sequences.

Also within the scope of the invention are antibodies, or antigen-binding fragments thereof, which bind overlapping epitopes of, or competitively inhibit, the binding of the anti-MUC1-H antibodies disclosed herein to MUC1-H, e.g., antibodies which bind overlapping epitopes of, or competitively inhibit, the binding of monospecific antibodies to MUC1-H. Any combination of anti-MUC1-H antibodies is within the scope of the invention, e.g., two or more antibodies that bind to different regions of MUC1-H, e.g., antibodies that bind to two different epitopes on MUC1-H, e.g., a bispecific antibody.

In one embodiment, the anti-MUC1-H antibody, or antigen-binding fragment thereof, includes at least one light or heavy chain immunoglobulin (or preferably, at least one light chain immunoglobulin and at least one heavy chain immunoglobulin). Preferably, each immunoglobulin includes a light or a heavy chain variable region having at least one, two and, preferably, three complementarity determining regions (CDR's) substantially identical to a CDR from an anti-MUC1-H light or heavy chain variable region, respectively.

In other embodiments, the light or heavy chain variable framework of the anti-MUC1-H antibody, or antigen-binding fragment thereof, includes at least one, two, three, four, five, six, seven, eight, nine, ten, fifteen, sixteen, or seventeen amino acid residues from a human light or heavy chain variable framework, e.g., a light or heavy chain variable framework residue from a human mature antibody, a human germline sequence, or a consensus sequence. In one embodiment, the amino acid residue from the human light chain variable framework is the same as the residue found at the same position in a human germline. Preferably, the amino acid residue from the human light chain variable framework is the most common residue in the human germline at the same position.

An anti-MUC1-H ligand described herein can be used alone, e.g., can be administered to a subject or used in vitro in non-derivatized or unconjugated forms. In other embodiments, the anti-MUC1-H ligand can be derivatized, modified or linked to another functional molecule, e.g., another peptide, protein, isotope, cell, or insoluble support. For example, the anti-MUC1-H ligand can be functionally linked (e.g., by chemical coupling, genetic fusion, non-covalent association or otherwise) to one or more other molecular entities, such as an antibody (e.g., if the ligand is an antibody to form a bispecific or a multispecific antibody), a toxin, a radioisotope, a therapeutic (e.g., a cytotoxic or cytostatic) agent or moiety, among others. For example, the anti-MUC1-H ligand can be coupled to a radioactive ion (e.g., an α-, γ-, or β-emitter), e.g., iodine (¹³¹I, or ¹²⁵I), yttrium (⁹⁰Y), lutetium (¹⁷⁷Lu), actinium (²²⁵Ac), rhenium (¹⁸⁶Re), or bismuth (²¹² or ²¹³Bi).

In another aspect, the invention provides, compositions, e.g., pharmaceutical compositions, which include a pharmaceutically acceptable carrier, excipient or stabilizer, and at least one of the anti-MUC1-H ligands (e.g., antibodies or fragments thereof) described herein. In one embodiment, the compositions, e.g., the pharmaceutical compositions, comprise a combination of two or more of the aforesaid anti-MUC1-H ligands.

In another aspect, the invention features a kit that includes an anti-MUC1-H antibody (or fragment thereof), e.g., an anti-MUC1-H antibody (or fragment thereof) as described herein, for use alone or in combination with other therapeutic modalities, e.g., a cytotoxic or labeling agent, e.g., a cytotoxic or labeling agent as described herein, along with instructions on how to use the MUC1-H antibody or the combination of such agents to treat, prevent or detect cancerous lesions.

The invention also features nucleic acid sequences that encode a heavy and light chain immunoglobulin or immunoglobulin fragment described herein. For example, the invention features, a first and second nucleic acid encoding a heavy and light chain variable region, respectively, of a anti-MUC1-H antibody molecule as described herein. In another aspect, the invention features host cells and vectors containing the nucleic acids of the invention.

In another aspect, the invention features, a method of producing a anti-MUC1-H antibody, or antigen-binding fragment thereof. The method includes: providing a first nucleic acid encoding a heavy chain variable region, e.g., a heavy chain variable region as described herein; providing a second nucleic acid encoding a light chain variable region, e.g., a light chain variable region as described herein; and expressing said first and second nucleic acids in a host cell under conditions that allow assembly of said light and heavy chain variable regions to form an antigen binding protein. The first and second nucleic acids can be linked or unlinked, e.g., expressed on the same or different vector, respectively.

The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell. For example, nucleic acids encoding the antibodies described herein can be expressed in a transgenic animal. In one embodiment, the nucleic acids are placed under the control of a tissue-specific promoter (e.g., a mammary specific promoter) and the antibody is produced in the transgenic animal. For example, the antibody molecule is secreted into the milk of the transgenic animal, such as a transgenic cow, pig, horse, sheep, goat or rodent.

The invention also features a method of treating, e.g., ablating or killing, a cell, e.g, a normal, benign or hyperplastic cell (e.g., a cell found in pulmonary, breast, renal, urothelial, colonic, prostatic, or hepatic cancer and/or metastasis). Methods of the invention include contacting the cell with a anti-MUC1-H ligand, in an amount sufficient to treat, e.g., ablate or kill, the cell. The ligand can include a cytotoxic entity. Methods of the invention can be used, for example, to treat or prevent a disorder, e.g., a cancerous (e.g., a malignant or metastatic disorder), or non-cancerous disorder (e.g., a benign or hyperplastic disorder) by administering to a subject a anti-MUC1-H ligand in an amount effective to treat or prevent such disorder.

The subject method can be used on cells in culture, e.g., in vitro or ex vivo. For example, cancerous or metastatic cells (e.g., pulmonary, breast, renal, urothelial, colonic, prostatic, or hepatic cancer or metastatic cells) can be cultured in vitro in culture medium and the contacting step can be effected by adding the anti-MUC1-H ligand to the culture medium. The method can be performed on cells (e.g., cancerous or metastatic cells) present in a subject, as part of an in vivo (e.g., therapeutic or prophylactic) protocol. For in vivo embodiments, the contacting step is effected in a subject and includes administering the anti-MUC1-H ligand to the subject under conditions effective to permit both binding of the ligand to the cell, and the treating, e.g., the killing or ablating of the cell.

The method of the invention can be used to treat or prevent cancerous disorders, e.g., including but are not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), and genitourinary tract (e.g., renal, urothelial cells), pharynx, as well as adenocarcinomas which include malignancies such as most colon cancers, rectal cancer, renal-cell carcinoma, liver cancer, non-small cell carcinoma of the lung, cancer of the small intestine and cancer of the esophagus. Metastatic lesions of the aforementioned cancers can also be treated or prevented using the methods and compositions of the invention.

The subject can be a mammal, e.g., a primate, preferably a higher primate, e.g., a human (e.g., a patient having, or at risk of, a disorder described herein, e.g., cancer).

The anti-MUC1-H antibody or fragment thereof, e.g., an anti-MUC1-H antibody or fragment thereof as described herein, can be administered to the subject systemically (e.g., orally, parenterally, subcutaneously, intravenously, intramuscularly, intraperitoneally, intranasally, transdermally, or by inhalation), topically, or by application to mucous membranes, such as the nose, throat and bronchial tubes.

The methods of the invention can further include the step of monitoring the subject, e.g., for a reduction in one or more of: a reduction in tumor size; reduction in cancer markers, e.g., levels of cancer specific antigen; reduction in the appearance of new lesions, e.g., in a bone scan; a reduction in the appearance of new disease-related symptoms; or decreased or stabilization of size of soft tissue mass; or any parameter related to improvement in clinical outcome. The subject can be monitored in one or more of the following periods: prior to beginning of treatment; during the treatment; or after one or more elements of the treatment have been administered. Monitoring can be used to evaluate the need for further treatment with the same anti-MUC1-H ligand or for additional treatment with additional agents. Generally, a decrease in one or more of the parameters described above is indicative of the improved condition of the subject.

The anti-MUC1-H ligand can be used alone in unconjugated form to thereby ablate or kill the MUC1-H-expressing cells. For example, if the ligand is an antibody, the ablation or killing can be mediated by an antibody-dependent cell killing mechanism such as complement-mediated cell lysis and/or effector cell-mediated cell killing. In other embodiments, the anti-MUC1-H ligand can be bound to a substance, e.g., a cytotoxic agent or moiety, effective to kill or ablate the MUC1-H-expressing cells. For example, the anti-MUC1-H ligand can be coupled to a radioactive ion (e.g., an α-, γ-, or β-emitter), e.g., iodine (¹³¹I or ¹²⁵I), yttrium (⁹⁰Y), lutetium (¹⁷⁷Lu), actinium (²²⁵Ac), or bismuth (²¹³Bi). The methods and compositions of the invention can be used in combination with other therapeutic modalities. In one embodiment, the methods of the invention include administering to the subject an anti-MUC1-H ligand, e.g., an anti-MUC1-H antibody or fragment thereof, in combination with a cytotoxic agent, in an amount effective to treat or prevent said disorder. The ligand and the cytotoxic agent can be administered simultaneously or sequentially. In other embodiments, the methods and compositions of the invention are used in combination with surgical and/or radiation procedures.

In another aspect, the invention features methods for detecting the presence of a MUC1-H protein, in a sample, in vitro (e.g., a biological sample, a tissue biopsy, e.g., a cancerous lesion). The subject method can be used to evaluate, e.g., diagnose or stage a disorder described herein, e.g., a cancerous disorder. The method includes: (i) contacting the sample (and optionally, a reference, e.g., control, sample) with an anti-MUC1-H ligand, as described herein, under conditions that allow interaction of the anti-MUC1-H ligand and the MUC1-H protein to occur; and (ii) detecting formation of a complex between the anti-MUC1-H ligand, and the sample (and optionally, the reference, e.g., control, sample). Formation of the complex is indicative of the presence of MUC1-H protein, and can indicate the suitability or need for a treatment described herein. E.g., a statistically significant change in the formation of the complex in the sample relative to the reference sample, e.g., the control sample, is indicative of the presence of MUC1-H in the sample.

In yet another aspect, the invention provides a method for detecting the presence of MUC1-H in vivo (e.g., in vivo imaging in a subject). The subject method can be used to evaluate, e.g., diagnose, localize, or stage a disorder described herein, e.g., a cancerous disorder. The method includes: (i) administering to a subject (and optionally a control subject) an anti-MUC1-H ligand (e.g., an antibody or antigen binding fragment thereof), under conditions that allow interaction of the anti-MUC1-H ligand and the MUC1-H protein to occur; and (ii) detecting formation of a complex between the ligand and MUC1-H, wherein a statistically significant change in the formation of the complex in the subject relative to the reference, e.g., the control subject or subject's baseline, is indicative of the presence of the MUC1-H.

In other embodiments, a method of diagnosing or staging a disorder as described herein (e.g., a cancerous disorder), is provided. The method includes: (i) identifying a subject having, or at risk of having, the disorder; (ii) obtaining a sample of a tissue or cell affected with the disorder; (iii) contacting said sample or a control sample with an anti-MUC1-H ligand, under conditions that allow interaction of the binding agent and the MUC1-H protein to occur, and (iv) detecting formation of a complex. A statistically significant increase in the formation of the complex between the ligand with respect to a reference sample, e.g., a control sample, is indicative of the disorder or the stage of the disorder.

Preferably, the anti-MUC1-H ligand used in the in vivo and in vitro diagnostic methods is directly or indirectly labeled with a detectable substance to facilitate detection of the bound or unbound binding agent. Suitable detectable substances include various enzymes, prosthetic groups, fluorescent materials, luminescent materials and radioactive materials. In one embodiment, the anti-MUC1-H ligand is coupled to a radioactive ion, e.g., indium (¹¹¹In), iodine (¹³¹I or ¹²⁵I), yttrium (⁹⁰Y), actinium (²²⁵Ac), bismuth (²¹³Bi), sulfur (³⁵S), carbon (¹⁴C), tritium (³H), rhodium (¹⁸⁸Rh), or phosphorous (³²P). In another embodiment, the ligand is labeled with an NMR contrast agent.

The invention also provides polypeptides and nucleic acids that encompass a range of amino acid and nucleic acid sequences.

As used herein, the term “substantially identical” (or “substantially homologous”) is used herein to refer to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. In the case of antibodies, the second antibody has the same specificity and has at least 50% of the affinity of the same.

Sequences similar or homologous (e.g., at least about 85% sequence identity) to the sequences disclosed herein are also part of the invention. In some embodiments, the sequence identity can be about 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol Biol. 48:444-453) algorithm which has been incorporated into the GAP program in the GCG software package (Accelerys, Cambridge, UK), using either a BLOSUM62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM62 scoring matrix with a gap penalty of 12, a gap extend penalty of 4, and a frameshift gap penalty of 5.

As used herein, the term “homologous” is synonymous with “similarity” and means that a sequence of interest differs from a reference sequence by the presence of one or more amino acid substitutions, although modest amino acid insertions or deletions may also be present. Presently preferred means of calculating degrees of homology or similarity to a reference sequence are through the use of BLAST algorithms (available from the National Center of Biotechnology Information (NCBI), National Institutes of Health, Bethesda, Md., USA), in each case, using the algorithm default or recommended parameters for determining significance of calculated sequence relatedness. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated herein by reference in its entirety. Aqueous and nonaqueous methods are described in that reference and either can be used. Specific hybridization conditions referred to herein are as follows: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and preferably (4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the binding agent polypeptides of the invention may have additional conservative or non-essential amino acid substitutions, which do not have a substantial effect on the polypeptide functions. Whether or not a particular substitution will be tolerated, i.e., will not adversely affect desired biological properties, such as binding activity, can be determined as described in Bowie et al. ((1990) Science 247:1306-1310). A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A “non-essential” amino acid residue is a residue that can be altered from the wild-type sequence of the binding agent, e.g., the antibody, without abolishing or more preferably, without substantially altering a biological activity, whereas an “essential” amino acid residue results in such a change.

Other features and advantages of the instant invention will become more apparent from the following detailed description and claims.

### BRIEF DESCRIPTION OF THE DRAWINGS

FIG. 1 is a representation of the MUC1 amino acid sequences (SEQ ID NO: 1). The MUC1-H (“Stubble”) sequence (SEQ ID NO: 2) is at amino acids 318-382, and the alternative Stubble protein (SEQ ID NO: 3), formed by the alternative cleavage site extends from amino acids 331-382 of the full-length MUC1 protein (SEQ ID NO: 1).

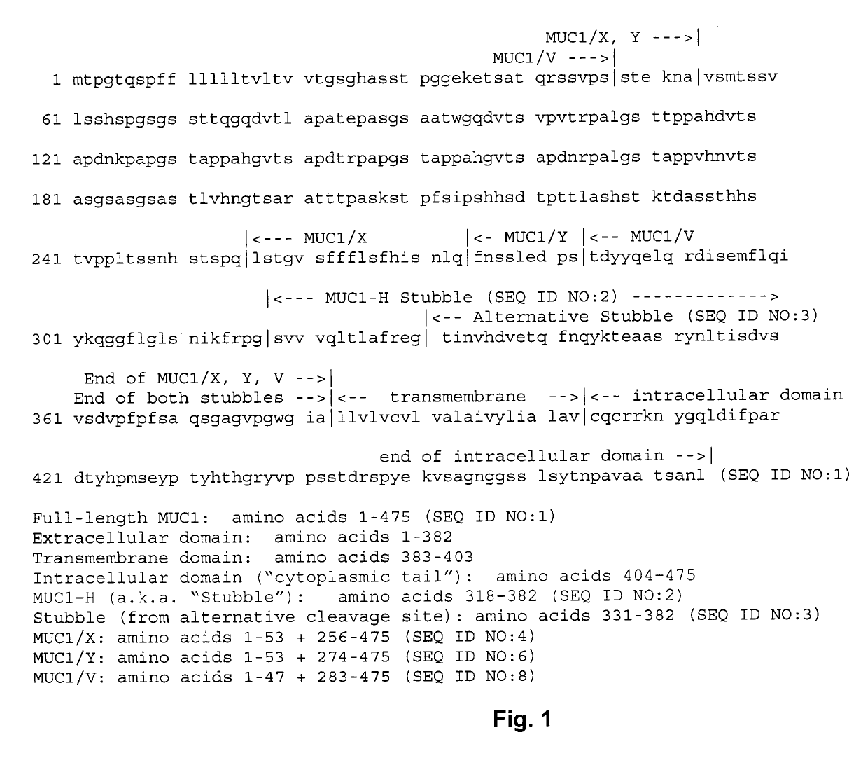

FIG. 2 is a representation of the MUC1/X and MUC1/X/alt amino acid sequences (SEQ ID NO: 4 and SEQ ID NO: 5, respectively).

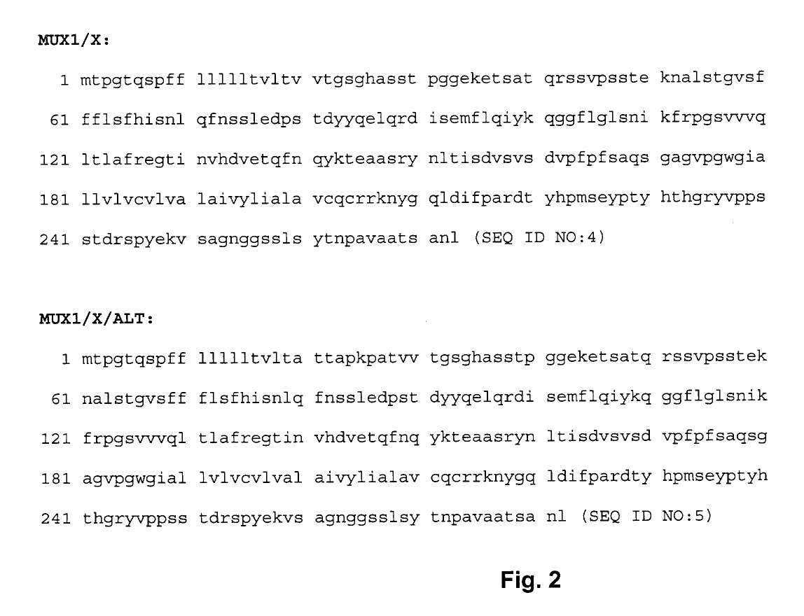

FIG. 3 is a representation of the MUC1/Y and MUC1/Y/alt amino acid sequences (SEQ ID NO: 6 and SEQ ID NO: 7, respectively).

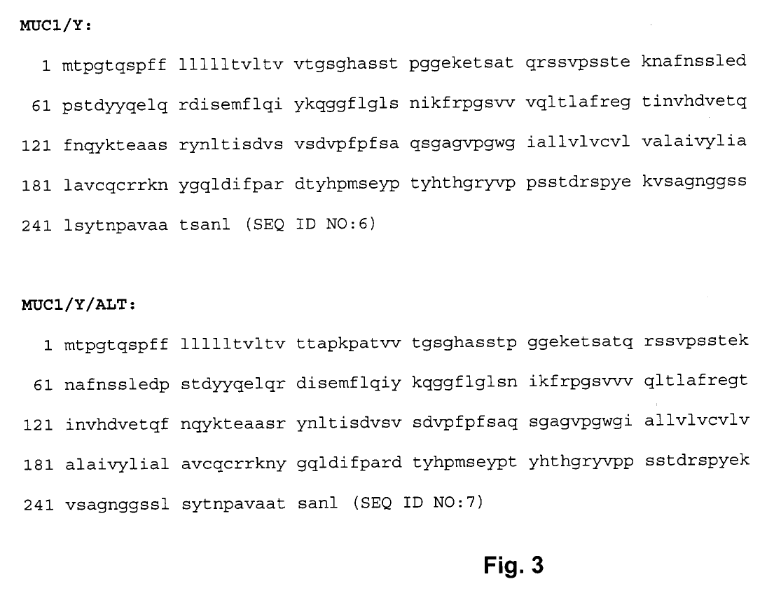

FIG. 4 is a representation of the MUC1/V and MUC1/V/alt (SEQ ID NO: 8 and SEQ ID NO: 9, respectively).

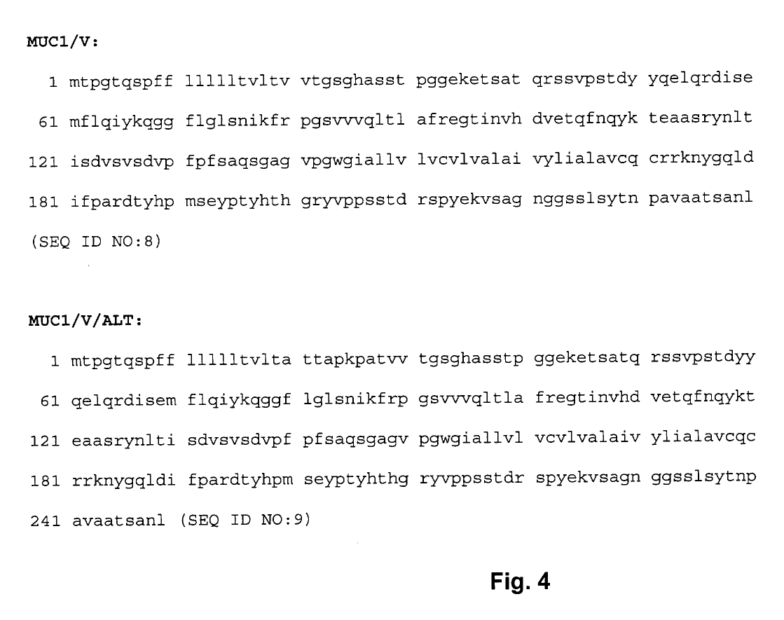

### DETAILED DESCRIPTION

Described herein are antibodies and ligands to MUC1-H, a group of epitopes present on cell-surface bound MUC1 but not on shed MUC1. Antibodies and ligands to MUC1-H recognize epitopes that are associated with the MUC1-stubble or the complex formed between soluble MUC1 and the MUC1-Stubble complex, and are thus devoid of binding with equivalent affinity to shed MUC1. MUC-H comprises epitopes that are hidden in full length, non-clipped MUC1 (or non-clipped splice variants of MUC1), and only appear on the cell-surface bound stump of MUC1 after specific cleavage of the MUC1 (or splice variants of MUC), hence the name MUC1-H(idden). Further, MUC1-H contains those epitopes that are formed between the shed form of MUC1 that remains associated with the MUC-stubble (possibly derived from an alternative splicing form of MUC1) and the MUC-stubble itself, such conformational epitopes are likely to be present only on cell-surface associated MUC1 and not on serum MUC1. Most of the hidden epitopes on MUC1-H will be based within the C-terminal 65 amino acids of the extracellular region of MUC1 (the MUC-Stubble), and can be linear or conformational epitopes within this sequence that are normally not detectable by a given polypeptide ligand on non-clipped MUC1 or splice variant of MUC1. Such epitopes can comprise the region on the MUC-Stubble that is responsible for the interaction with the shed form of MUC1 (such as the region encoded by the SEA domain). When such epitopes are normally covered due to interaction with shed MUC1, upon release of the latter they become exposed. Other MUC1-H epitopes comprise conformational epitopes formed by the complex of the N-terminal region of MUC1 after clipping at the junction with the Stubble, and the stubble itself; in such case the sequence will contain at least part of the C-terminal 65 amino acids of the extracellular region of MUC1. MUC1-H epitopes may be formed by a linear or conformational sequence withing the representative sequence of the MUC1-Stubble as is set out in SEQ ID NO: 2. In a preferred embodiment, the polypeptide ligands are antibodies (the term of which includes antigen-binding fragments). In another preferred embodiment, the polypeptide ligands are modified scaffold polypeptides or peptides. In still another preferred embodiment, the peptides (e.g., polypeptides of 6-25 amino acids) are cyclic peptides or linear peptides. A polypeptide ligand may be a multi-chain protein (e.g., including at least two peptides or polypeptides). An example of a multi-chain protein is an antibody. Whereas many examples described herein refer to antibody ligands, it is understood, that the invention can be practiced using any polypeptide ligand (e.g., antibody and non-antibody ligand) provided herein.

The invention provides, in part, methods for identifying proteins that bind to MUC1-H. In many cases, the identified proteins are at least partially specific.

In MUC1-H the SEA domain is partially cleaved off, allowing structural differences as compared with the complete MUC1 molecule.

In MUC1-H the SEA domain is partially cleaved off, allowing structural differences as compared with the complete MUC1 molecule.

MUC1-H epitopes are different from MUC1. For therapy, internalization can be important for delivery of toxic drugs, radioactive labels, etc. Therefore, the polypeptide ligands of the present invention are preferably also internalized with the MUC1-H, which permits the intracellular delivery of an agent conjugated to the antibody, e.g., a cytotoxic or a labeling agent. Accordingly, the polypeptide ligands of the invention can be used to target living normal, benign hyperplastic, and cancerous cells that express the MUC1-H epitopes. Such internalization may be based on the natural internalization of the bound MUC1 protein variant, or may be enhanced or created by signals provide on the polypeptide ligand. Antibodies and peptides against MUC1-H can therefore be used to target tumors in research, diagnostics and therapy.

The methods include providing a library and screening the library to identify a member that encodes a protein that binds to the MUC1-H.

The screening can be performed in a number of ways. For example, the library can be a display library. The library can be a cyclic peptide library, e.g., like the libraries described in U.S. Pat. No. 6,197,526 B1, which is incorporated herein by reference in its entirety.

The MUC1-H can be tagged and recombinantly expressed. The MUC1-H can be purified and attached to a support, e.g., to paramagnetic beads or other magnetically responsive particle.

The MUC1-H can also be expressed on the surface of a cell. The display library can be selected on cells that express MUC1-H. The display library can be screened to identify members that specifically bind to the cell, e.g., only if the MUC1-H is expressed.

A. Peptide Libraries

Peptide ligands which bind to MUC1-H are also included herein. The term “peptide ligand” or “binding molecule” as used herein refers to any molecule, polypeptide, peptidomimetic or transformed cell (“transformant”) capable of forming a binding complex with another molecule, polypeptide, peptidomimetic or tranformant. A “MUC1-H peptide ligand” or “MUC1-H binding molecule” is a binding molecule that forms a complex with MUC1-H. Also included within the definition of MUC1-H peptide ligands are such polypeptides which have been modified for particular results. Specific examples of modifications contemplated are C-terminal or N-terminal amino acid substitutions or polypeptide chain elongations for the purpose of linking the binding moiety to a chromatographic support or other substrate, and substitutions of pairs of cysteine residues that normally form disulfide links, for example with non-naturally occurring amino acid residues having reactive side chains, for the purpose of forming a more stable bond between those amino acid positions than the former disulfide bond. All such modified binding molecules are also considered binding molecules according to this invention so long as they retain the ability to bind MUC1-H and/or MUC1-H-like polypeptides.

1. Selecting Binding and Release Conditions

Polypeptide binding molecules according to the present invention are isolated using phage display technology, in a manner to identify MUC1-H binding peptides exhibiting particular preselected properties of binding and release. According to this methodology, two solution conditions may be preselected, i.e., binding conditions and release conditions. The binding conditions are a set of solution conditions under which it is desired that a discovered binding polypeptide will bind the target MUC1-H (or MUC1-H-like polypeptide); the release conditions are a set of solution conditions under which it is desired that a discovered binding polypeptide will not bind the MUC1-H (i.e., will dissociate from MUC1-H). The two conditions may be selected to satisfy any criterion of the practitioner, such as ease of attaining the conditions, compatibility with other purification steps, lowered cost of switching between conditions compared to other affinity media, etc. Preferably, the two solution conditions are selected so as not to adversely affect the stability or activity of the target protein (MUC1-H or MUC1-H-like polypeptide) and so as to differ significantly with respect to at least one solution parameter. For example, in conducting the screening for suitable binding peptides described herein, binders are selected that dissociated from the target in the presence of an ethylene glycol-containing buffer, or conditions of lowered pH (i.e., pH 2), or combinations of those conditions, which differ from the conditions employed for binding. Another parameter that can be advantageously varied is the concentration of a salt, for example NaCl, in the binding and elution buffers.

2. Selection of a Parental Binding Domain

In conjunction with selecting specific solution conditions for the desired binding and release of MUC1-H, a parental binding domain is selected to serve as a structural template for the engineered binding molecules that will exhibit the desired binding and release capabilities. The binding domain may be a naturally occurring or synthetic protein, or a region or domain of a protein. The parental binding domain may be selected based on knowledge of a known interaction between the parental binding domain and MUC1-H, but this is not critical. In fact, it is not essential that the parental binding domain have any affinity for MUC1-Hat all: its purpose is to provide a structure from which a multiplicity of analogues (a “library”) can be generated, which multiplicity of analogues will include one or more analogues that exhibit the desired binding and release properties (and any other properties selected for). The binding conditions and the release conditions discussed supra may be selected with knowledge of the exact polypeptide that will serve as the parental binding domain, or with knowledge of a class of proteins or domains to which the parental binding domain belongs, or completely independently of the choice of the parental binding domain. Similarly, the binding and/or release conditions may be selected with regard to known interactions between a binding domain and MUC1-H, e.g., to favor the interaction under one or both of the solution conditions, or they may be selected without regard to such known interactions. Likewise, the parental binding domain can be selected taking into account the binding and/or release conditions or not, although it must be recognized that if the binding domain analogues are unstable under the binding or release conditions, no useful binding molecules may be obtained.

The nature of the parental binding domain greatly influences the properties of the derived peptides (analogues) that will be tested against MUC1-H molecule. In selecting the parental binding domain, the most important consideration is how the analogue domains will be presented to MUC1-H, i.e., in what conformation MUC1-H and the analogues will come into contact. In preferred embodiments, for example, the analogues will be generated by insertion of synthetic DNA encoding the analogue into a replicable genetic package, resulting in display of the domain on the surface of a microorganism, such as M13 phage, using techniques as described, e.g., in Kay et al., Phage Display of Peptides and Proteins: A Laboratory Manual, (Academic Press, Inc.; San Diego 1996) and U.S. Pat. No. 5,223,409 (Ladner et al.), both of whch are incorporated herein by reference in their entirety.

For formation of phage display libraries, it is preferred to use structured polypeptides as the binding domain template, as opposed to unstructured, linear peptides. Mutation of surface residues in a protein will usually have little effect on the overall structure or general properties (such as size, stability, and temperature of denaturation) of the protein; while at the same time mutation of surface residues may profoundly affect the binding properties of the protein. The more tightly a peptide segment is constrained, the less likely it is to bind to any particular target. If it does bind, however, the binding is likely to be tighter and more specific. Thus, it is preferred to select a parental binding domain and, in turn, a structure for the polypeptide analogues, that is constrained within a framework having some degree of rigidity.

Preferably the protein domain that is used as the template or parental domain for generating the library of domain analogues will be a small protein or polypeptide. Small proteins or polypeptides offer several advantages over large proteins. First, the mass per binding site is reduced. Highly stable protein domains having low molecular weights, e.g., Kunitz domains (˜7 kDa), Kazal domains (˜7 kDa), Cucurbita maxima trypsin inhibitor (CMTI) domains (˜3.5 kDa), and endothelin (˜2 kDa), can show much higher binding per gram than do antibodies (150 kDa) or single-chain antibodies (30 kDa). Second, the possibility of non-specific binding is reduced because there is less surface available. Third, small proteins or polypeptides can be engineered to have unique tethering sites in a way that is impracticable for larger proteins or antibodies. For example, small proteins can be engineered to have lysines only at sites suitable for tethering (e.g., to a chromatography matrix), but this is not feasible for antibodies. Fourth, a constrained polypeptide structure is more likely to retain its functionality when transferred with the structural domain intact from one framework to another. For instance, the binding domain structure is likely to be transferable from the framework used for presentation in a library (e.g., displayed on a phage) to an isolated protein removed from the presentation framework or immobilized on a chromatographic substrate.

Immobilization of the polypeptides according to the invention is contemplated, e.g., onto chromatographic matrices to form efficient MUC1-H separation media for solutions such as whole blood or conditioned culture media containing MUC1-H secreted from a transformant host cell. By selecting appropriate binding domain templates, binding polypeptides having a single free (unpaired with another cysteine that ordinarily forms a disulfide link) cysteine can be isolated. Such thiol-functional polypeptides can be used for highly stable immobilization to substrates by formation of a thioether with iodoacetamide, iodoacetic acid, or similar α-iodo carboxylic acid groups.

Similarly, the C-terminal carboxyl group of the polypeptide domain may be converted to a hydrazide (—NH—NH₂), for reaction with an aldehyde-functional substrate or other amine-reactive substrate. This technique is preferred.

There are many small, stable protein domains suitable for use as parental binding domains and for which the following useful information is available: (1) amino acid sequence, (2) sequences of several homologous domains, (3) 3-dimensional structure, and/or (4) stability data over a range of pH, temperature, salinity, organic solvent, oxidant concentration. Some examples are: Kunitz domains (58 amino acids, 3 disulfide bonds), Cucurbita maxima trypsin inhibitor domains (31 amino acids, 3 disulfide bonds), domains related to guanylin (14 amino acids, 2 disulfide bonds), domains related to heat-stable enterotoxin IA from gram negative bacteria (18 amino acids, 3 disulfide bonds), EGF domains (50 amino acids, 3 disulfide bonds), kringle domains (60 amino acids, 3 disulfide bonds), fungal carbohydrate-binding domains (35 amino acids, 2 disulfide bonds), endothelin domains (18 amino acids, 2 disulfide bonds), and Streptococcal G IgG-binding domain (35 amino acids, no disulfide bonds). Most but not all of these contain disulfide bonds that maintain and stabilize the structure. The parental binding domain may also be based on a single loop (one disulfide) of a microprotein that is homologous to a known protein domain or not. For example, constrained loops of 7 to 9 amino acids were used to form libraries for isolating MUC1-H and MUC1-H-like polypeptide binding molecules. Libraries based on these domains, preferably displayed on phage, can be readily constructed and used for the selection of binding molecules according to this invention.

3. Providing a Library of Parental Binding Domain Analogues

Once a parental binding domain has been selected, a library of potential binding molecules is created for screening against a target, in this case MUC1-H and/or MUC1-H-like proteins, under the desired binding conditions and (optionally) the desired elution (release) conditions. The library is created by making a series of analogues, each analogue corresponding to the parental binding domain except having one or more amino acid substitutions in the amino acid sequence of the domain. The amino acid substitutions are expected to alter the binding properties of the domain without significantly altering its structure, at least for most substitutions. It is preferred that the amino acid positions that are selected for variation (variable amino acid positions) will be surface amino acid positions, that is, positions in the amino acid sequence of the domains which, when the domain is in its most stable conformation, appear on the outer surface of the domain (i.e., the surface exposed to solution). Most preferably the amino acid positions to be varied will be adjacent or close together, so as to maximize the effect of substitutions. In addition, extra amino acids can be added into the structure of the parental binding domain. In preferred embodiments, especially where a great deal of information is available concerning the interactions of MUC1-H with other molecules, particularly the parental binding domain, those amino acid positions that are essential to binding interactions will be determined and conserved in the process of building the analogue library (i.e., the amino acids essential for binding will not be varied).

The object of creating the analogue library is to provide a very large number of potential binding molecules for reaction with the MUC1-H molecule, and in general the greater the number of analogues in the library, the greater the likelihood that at least one member of the library will bind to MUC1-H and release under preselected or desirable conditions for release. Designed libraries following a particular template structure and limiting amino acid variegation at particular positions are much preferred, since a single library can encompass all the designed analogues and the included sequences will be known and presented in roughly equal numbers. By contrast, random substitution at only six positions in an amino acid sequence provides over 60 million analogues, which is a library size that begins to present practical limitations even when utilizing screening techniques as powerful as phage display. Libraries larger than this would pose problems in handling, e.g., fermentation vessels would need to be of extraordinary size, and more importantly, the likelihood of having all of the planned polypeptide sequence variations represented in the prepared library would decrease sharply. It is therefore preferred to create a designed or biased library, in which the amino acid positions designated for variation are considered so as to maximize the effect of substitution on the binding characteristics of the analogue, and the amino acid residues allowed or planned for use in substitutions are limited, e.g., on the basis that they are likely to cause the analogue to bind under the solution conditions at which the library will be screened for binders.

As indicated previously, the techniques discussed in Kay et al., supra, and Ladner et al., U.S. Pat. No. 5,223,409 are particularly useful in preparing a library of analogues corresponding to a selected parental binding domain, which analogues will be presented in a form suitable for large-scale screening of large numbers of analogues with respect to a target MUC1-H molecule. The use of replicable genetic packages, and most preferably bacteriophage, is a powerful method of generating novel polypeptide binding entities that involves introducing a novel, exogenous DNA segment into the genome of a bacteriophage (or other amplifiable genetic package) so that the polypeptide encoded by the non-native DNA appears on the surface of the phage. When the inserted DNA contains sequence diversity, then each recipient phage displays one variant of the template (parental) amino acid sequence encoded by the DNA, and the phage population (library) displays a vast number of different but related amino acid sequences.

In a screening procedure to obtain MUC1-H binders according to this invention, a phage library is contacted with and allowed to bind a target MUC1-H molecule, usually immobilized on a solid support. Non-binders are separated from binders. In various ways, the bound phage are liberated from the MUC1-H, collected and amplified. Since the phage can be amplified through infection of bacterial cells, even a few binding phage are sufficient to reveal the gene sequence that encodes a binding entity. Using these techniques it is possible to recover a binding phage that is about 1 in 20 million in the population. One or more libraries, displaying 10-20 million or more potential binding polypeptides each, can be rapidly screened to find high-affinity MUC1-H binders. When the selection process works, the diversity of the population falls with each round until only good binders remain, i.e., the process converges. Typically, a phage display library will contain several closely related binders (10 to 50 binders out of 10 million). Indications of convergence include increased binding (measured by phage titers) and recovery of closely related sequences. After a first set of binding peptides is identified, the sequence information can be used to design other libraries biased for members having additional desired properties, e.g., discrimination between MUC1-H and particular fragments or closely related impurities in a particular feed stream.

Such techniques make it possible not only to screen a large number of potential binding molecules but make it practical to repeat the binding/elution cycles and to build secondary, biased libraries for screening analog-displaying packages that meet initial criteria. Using these techniques, an analogue biased library may be screened to reveal members that bind tightly (i.e., with high affinity) under the screening conditions.

4. Synthesis of Polypeptide Analogues

Following the procedures outlined above, additional binding molecules for MUC1-H and/or MUC1-H-like polypeptides may be isolated from the phage display libraries described herein or other phage display libraries or collections of potential binding molecules (e.g., combinatorial libraries of organic compounds, random peptide libraries, etc.). Once isolated, the sequence of any individual binding peptide or the structure of any binding molecule can be analyzed, and the binder may be produced in any desired quantity using known methods. For example, the polypeptide binding molecules described herein, since their sequences are now known, may advantageously be produced by chemical synthesis followed by treatment under oxidizing conditions appropriate to obtain the native conformation, i.e., the correct disulfide bond linkages. Synthesis may be carried out by methodologies well known to those skilled in the art (see, Kelley et al., in Genetic Engineering Principles and Methods, (Setlow, J. K., ed.), Plenum Press, NY., (1990) vol. 12, pp. 1-19; Stewart et al., Solid-Phase Peptide Synthesis (1989), W. H. Freeman Co., San Francisco). The binding molecules of the present invention can be made either by chemical synthesis or by semisynthesis. The chemical synthesis or semisynthesis methods allow the possibility of non-natural amino acid residues to be incorporated.

Polypeptide binding molecules of the present invention are preferably prepared using solid phase peptide synthesis (Merrifield (1963) J. Am. Chem. Soc. 85:2149; Houghten (1985) Proc. Natl. Acad. Sci. USA 82:5132). Solid phase synthesis begins at the carboxy-terminus of the putative polypeptide by coupling a protected amino acid to a suitable resin, which reacts with the carboxy group of the C-terminal amino acid to form a bond that is readily cleaved later, such as a halomethyl resin, e.g., chloromethyl resin and bromomethyl resin, hydroxymethyl resin, aminomethyl resin, benzhydrylamine resin, or t-alkyloxycarbonyl-hydrazide resin. After removal of the α-amino protecting group with, for example, trifluoroacetic acid (TFA) in methylene chloride and neutralizing in, for example, TEA, the next cycle in the synthesis is ready to proceed. The remaining α-amino and, if necessary, side-chain-protected amino acids are then coupled sequentially in the desired order by condensation to obtain an intermediate compound connected to the resin. Alternatively, some amino acids may be coupled to one another forming an oligopeptide prior to addition of the oligopeptide to the growing solid phase polypeptide chain.

The condensation between two amino acids, or an amino acid and a peptide, or a peptide and a peptide can be carried out according to the usual condensation methods such as azide method, mixed acid anhydride method, DCC (dicyclohexylcarbodiimide) method, active ester method (p-nitrophenyl ester method, BOP [benzotriazole-1-yl-oxy-tris(dimethylamino)phosphonium hexafluorophosphate] method, N-hydroxysuccinic acid imido ester method), and Woodward reagent K method.

Common to chemical synthesis of peptides is the protection of the reactive side-chain groups of the various amino acid moieties with suitable protecting groups at that site until the group is ultimately removed after the chain has been completely assembled. Also common is the protection of the α-amino group on an amino acid or a fragment while that entity reacts at the carboxyl group followed by the selective removal of the α-amino -protecting group to allow subsequent reaction to take place at that location. Accordingly, it is common that, as a step in the synthesis, an intermediate compound is produced which includes each of the amino acid residues located in the desired sequence in the polypeptide chain with various of these residues having side-chain protecting groups. These protecting groups are then commonly removed substantially at the same time so as to produce the desired resultant product following purification.

The typical protective groups for protecting the α- and ε-amino side chain groups are exemplified by benzyloxycarbonyl (Z), isonicotinyloxycarbonyl (iNOC), O-chlorobenzyloxycarbonyl[Z(NO₂)], p-methoxybenzyloxycarbonyl[Z(OMe)], t-butoxycarbonyl (Boc), t-amyioxycarbonyl (Aoc), isobornyloxycarbonyl, adamatyloxycarbonyl, 2-(4-biphenyl)-2-propyloxycarbonyl (Bpoc), 9-fluorenylmethoxycarbonyl (Fmoc), methylsulfonyiethoxycarbonyl (Msc), trifluoroacetyl, phthalyl, formyl, 2-nitrophenylsulphenyl (NPS), diphenylphosphinothioyl (Ppt), dimethylophosphinothioyl (Mpt), and the like.

As protective groups for the carboxy group there can be exemplified, for example, benzyl ester (OBzl), cyclohexyl ester (Chx), 4-nitrobenzyl ester (ONb), t-butyl ester (Obut), 4-pyridylmethyl ester (OPic), and the like. It desirable that specific amino acids such as arginine, cysteine, and serine possessing a functional group other than amino and carboxyl groups are protected by a suitable protective group as occasion demands. For example, the guanidino group in arginine may be protected with nitro, p-toluenesulfonyl, benzyloxycarbonyl, adamantyloxycarbonyl, p-methoxybenzenesulfonyl, 4-methoxy-2,6-dimethylbenzenesulfonyl (Mds), 1,3,5-trimethylphenysulfonyl (Mts), and the like. The thiol group in cysteine may be protected with p-methoxybenzyl, triphenylmethyl, acetylaminomethyl ethylcarbamoyl, 4-methylbenzyl, 2,4,6-trimethy-benzyl (Tmb), etc., and the hydroxyl group in the serine can be protected with benzyl, t-butyl, acetyl, tetrahydropyranyl, etc.

After the desired amino acid sequence has been completed, the intermediate polypeptide is removed from the resin support by treatment with a reagent, such as liquid HF and one or more thio-containing scavengers, which not only cleaves the polypeptide from the resin, but also cleaves all the remaining side-chain protecting groups. Following HF cleavage, the protein sequence is washed with ether, transferred to a large volume of dilute acetic acid, and stirred at pH adjusted to about 8.0 with ammonium hydroxide. Upon pH adjustment, the polypeptide takes its desired conformational arrangement.

Polypeptides according to the invention may also be prepared commercially by companies providing polypeptide synthesis as a service (e.g., BACHEM Bioscience, Inc., King of Prussia, Pa.; Quality Controlled Biochemicals, Inc., Hopkinton, Mass.).

B. Display Libraries

A display library is used to identify proteins that bind to the MUC1-H. A display library is a collection of entities; each entity includes an accessible polypeptide component and a recoverable component that encodes or identifies the peptide component. The polypeptide component can be of any length, e.g., from three amino acids to over 300 amino acids. In a selection, the polypeptide component of each member of the library is probed with the MUC1-H and if the polypeptide component binds to the MUC1-H, the display library member is identified, typically by retention on a support.

Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

A variety of formats can be used for display libraries. Examples include the following.

1. Phage Display

One format utilizes viruses, particularly bacteriophages. This format is termed “phage display.” The peptide component is typically covalently linked to a bacteriophage coat protein. The linkage results from translation of a nucleic acid encoding the peptide component fused to the coat protein. The linkage can include a flexible peptide linker, a protease site, or an amino acid incorporated as a result of suppression of a stop codon. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem. 274:18218-30; Hoogenboom et al. (1998) Immunotechnology 4:1-20; Hoogenboom et al. (2000) Immunol. Today 2:371-8; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Bio/Technology 9:1373-1377; Rebar et al. (1996) Methods Enzymol. 267:129-49; Hoogenboom et al. (1991) Nuc. Acids Res. 19:4133-4137; and Barbas et al. (1991) Proc. Natl. Acad. Sci. USA 88:7978-7982.

Phage display systems have been developed for filamentous phage (phage fl, fd, and M13) as well as other bacteriophage (e.g., T7 bacteriophage and lambdoid phages; see, e.g., Santini (1998) J. Mol. Biol. 282:125-135; Rosenberg et al. (1996) Innovations 6:1-6; Houshmet et al. (1999) Anal. Biochem. 268:363-370). The filamentous phage display systems typically use fusions to a minor coat protein, such as gene III protein, and gene VIII protein, a major coat protein, but fusions to other coat proteins such as gene VI protein, gene VII protein, gene IX protein, or domains thereof can also been used (see, e.g., WO 00/71694). In a preferred embodiment, the fusion is to a domain of the gene III protein, e.g., the anchor domain or “stump,” (see, e.g., U.S. Pat. No. 5,658,727 for a description of the gene III protein anchor domain).

The valency of the peptide component can also be controlled. Cloning of the sequence encoding the peptide component into the complete phage genome results in multivariant display since all replicates of the gene III protein are fused to the peptide component. For reduced valency, a phagemid system can be utilized. In this system, the nucleic acid encoding the peptide component fused to gene III is provided on a plasmid, typically of length less than 700 nucleotides. The plasmid includes a phage origin of replication so that the plasmid is incorporated into bacteriophage particles when bacterial cells bearing the plasmid are infected with helper phage, e.g., M13K01. The helper phage provides an intact copy of gene III and other phage genes required for phage replication and assembly. The helper phage has a defective origin such that the helper phage genome is not efficiently incorporated into phage particles relative to the plasmid that has a wild type origin.

Bacteriophage displaying the peptide component can be grown and harvested using standard phage preparatory methods, e.g., PEG precipitation from growth media.

After selection of individual display phages, the nucleic acid encoding the selected peptide components, by infecting cells using the selected phages. Individual colonies or plaques can be picked, the nucleic acid isolated and sequenced.

2. Cell-Based Display

In still another format the library is a cell-display library. Proteins are displayed on the surface of a cell, e.g., a eukaryotic or prokaryotic cell. Exemplary prokaryotic cells include E. coli cells, B. subtilis cells, spores (see, e.g., Lu et al. (1995) Biotechnology 13:366). Exemplary eukaryotic cells include yeast (e.g., Saccharomyces cerevisiae, Schizosaccharomyces pombe, Hanseula, or Pichia pastoris). Yeast surface display is described, e.g., in Boder and Wittrup (1997) Nature Biotech. 15:553-557 and U.S. Provisional Patent Application No. Serial No. 60/326,320, filed Oct. 1, 2001, titled “Multi-Chain Eukaryotic Display Vectors And The Uses Thereof.” This application describes a yeast display system that can be used to display immunoglobulin proteins such as Fab fragments, and the use of mating to generate combinations of heavy and light chains.

In one embodiment, variegated nucleic acid sequences are cloned into a vector for yeast display. The cloning joins the variegated sequence with a domain (or complete) yeast cell surface protein, e.g., Aga2, Aga1, Flo1, or Gas1. A domain of these proteins can anchor the polypeptide encoded by the variegated nucleic acid sequence by a transmembrane domain (e.g., Flo1) or by covalent linkage to the phospholipid bilayer (e.g., Gas1). The vector can be configured to express two polypeptide chains on the cell surface such that one of the chains is linked to the yeast cell surface protein. For example, the two chains can be immunoglobulin chains.

3. Ribosome Display

RNA and the polypeptide encoded by the RNA can be physically associated by stabilizing ribosomes that are translating the RNA and have the nascent polypeptide still attached. Typically, high divalent Mg²⁺ concentrations and low temperature are used. See, e.g., Mattheakis et al. (1994) Proc. Natl. Acad. Sci. USA 91:9022 and Hanes et al. (2000) Nature Biotech. 18:1287-92; Hanes et al. (2000) Methods Enzymol. 328:404-30. and Schaffitzel et al. (1999) J. Immunol. Methods 231:119-35.

4. Peptide-Nucleic Acid Fusions

Another format utilizes peptide-nucleic acid fusions. Polypeptide-nucleic acid fusions can be generated by the in vitro translation of mRNA that include a covalently attached puromycin group, e.g., as described in Roberts and Szostak (1997) Proc. Natl. Acad. Sci. USA 94:12297-12302, and U.S. Pat. No. 6,207,446. The mRNA can then be reverse transcribed into DNA and crosslinked to the polypeptide.

5. Other Display Formats

Yet another display format is a non-biological display in which the polypeptide component is attached to a non-nucleic acid tag that identifies the polypeptide. For example, the tag can be a chemical tag attached to a bead that displays the polypeptide or a radiofrequency tag (see, e.g., U.S. Pat. No. 5,874,214).

6. Scaffolds

Scaffolds for display can include: antibodies (e.g., Fab fragments, single chain Fv molecules (scFV), single domain antibodies, camelid antibodies, and camelized antibodies); T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains).

Appropriate criteria for evaluating a scaffolding domain can include: (1) amino acid sequence, (2) sequences of several homologous domains, (3) three-dimensional structure, and/or (4) stability data over a range of pH, temperature, salinity, organic solvent, oxidant concentration. In one embodiment, the scaffolding domain is a small, stable protein domain, e.g., a protein of less than 100, 70, 50, 40 or 30 amino acids. The domain may include one or more disulfide bonds or may chelate a metal, e.g., zinc.

Examples of small scaffolding domains include: Kunitz domains (58 amino acids, 3 disulfide bonds), Cucurbita maxima trypsin inhibitor domains (31 amino acids, 3 disulfide bonds), domains related to guanylin (14 amino acids, 2 disulfide bonds), domains related to heat-stable enterotoxin IA from gram negative bacteria (18 amino acids, 3 disulfide bonds), EGF domains (50 amino acids, 3 disulfide bonds), kringle domains (60 amino acids, 3 disulfide bonds), fungal carbohydrate-binding domains (35 amino acids, 2 disulfide bonds), endothelin domains (18 amino acids, 2 disulfide bonds), and Streptococcal G IgG-binding domain (35 amino acids, no disulfide bonds).

Examples of small intracellular scaffolding domains include SH2, SH3, and EVH domains. Generally, any modular domain, intracellular or extracellular, can be used.

Another useful type of scaffolding domain is the immunoglobulin (Ig) domain. Methods using immunoglobulin domains for display are described below (see, e.g., “Antibody Display Libraries”).

Display technology can also be used to obtain ligands, e.g., antibody ligands, particular epitopes of a target. This can be done, for example, by using competing non-target molecules that lack the particular epitope or are mutated within the epitope, e.g., with alanine. Such non-target molecules can be used in a negative selection procedure as described below, as competing molecules when binding a display library to the target, or as a pre-elution agent, e.g., to capture in a wash solution dissociating display library members that are not specific to the target.

7. Iterative Selection

In one preferred embodiment, display library technology is used in an iterative mode. A first display library is used to identify one or more ligands for a target. These identified ligands are then varied using a mutagenesis method to form a second display library. Higher affinity ligands are then selected from the second library, e.g., by using higher stringency or more competitive binding and washing conditions.

In some implementations, the mutagenesis is targeted to regions known or likely to be at the binding interface. If, for example, the identified ligands are antibodies, then mutagenesis can be directed to the CDR regions of the heavy or light chains as described herein. Further, mutagenesis can be directed to framework regions near or adjacent to the CDRs. In the case of antibodies, mutagenesis can also be limited to one or a few of the CDRs, e.g., to make precise step-wise improvements. Likewise, if the identified ligands are enzymes, mutagenesis can be directed to the active site and vicinity.

Some exemplary mutagenesis techniques include: error-prone PCR (Leung et al. (1989) Technique 1:11-15), recombination, DNA shuffling using random cleavage (Stemmer (1994) Nature 389-391; termed “nucleic acid shuffling”), RACHITT™ (Coco et al. (2001) Nature Biotech. 19:354), site-directed mutagenesis (Zooler et al (1987) Nucl. Acids Res. 10:6487-6504), cassette mutagenesis (Reidhaar-Olson (1991) Methods Enzymol. 208:564-586) and incorporation of degenerate oligonucleotides (Griffiths et al. (1994) EMBO J. 13:3245).

In one example of iterative selection, the methods described herein are used to first identify a polypeptide ligand from a display library that binds a MUC1-H with at least a minimal binding specificity for a target or a minimal activity, e.g., an equilibrium dissociation constant for binding of greater than 1 nM, 10 nM, or 100 nM. The nucleic acid sequence encoding the initial identified polypeptide ligand is used as a template nucleic acid for the introduction of variations, e.g., to identify a second polypeptide ligand that has enhanced properties (e.g., binding affinity, kinetics, or stability) relative to the initial polypeptide ligand.

8. Off-Rate Selection

Since a slow dissociation rate can be predictive of high affinity, particularly with respect to interactions between polypeptides and their targets, the methods described herein can be used to isolate ligands with a desired kinetic dissociation rate (i.e., reduced) for a binding interaction to a target.

To select for slow dissociating ligands from a display library, the library is contacted to an immobilized target. The immobilized target is then washed with a first solution that removes non-specifically or weakly bound biomolecules. Then the immobilized target is eluted with a second solution that includes a saturation amount of free target, i.e., replicates of the target that are not attached to the particle. The free target binds to biomolecules that dissociate from the target. Rebinding is effectively prevented by the saturating amount of free target relative to the much lower concentration of immobilized target.

The second solution can have solution conditions that are substantially physiological or that are stringent. Typically, the solution conditions of the second solution are identical to the solution conditions of the first solution. Fractions of the second solution are collected in temporal order to distinguish early from late fractions. Later fractions include biomolecules that dissociate at a slower rate from the target than biomolecules in the early fractions.

Further, it is also possible to recover display library members that remain bound to the target even after extended incubation. These can either be dissociated using chaotropic conditions or can be amplified while attached to the target. For example, phage bound to the target can be contacted to bacterial cells.

9. Selecting or Screening for Specificity

The display library screening methods described herein can include a selection or screening process that discards display library members that bind to a non-target molecule. Examples of non-target molecules include: (i) full-length MUC1 protein; (ii) MUC1/X protein; (iii) MUC1/X/alt protein; (iv) MUC1/Y protein; (v) MUC1/Y/alt protein; (vi) MUC1/V protein; (vii) MUC1/V/alt protein; (viii) MUC1/W protein; (ix) MUC1/W/alt protein; (x) MUC1/Z protein; or (xi) MUC1/Z/alt protein.

In one implementation, a so-called “negative selection” step is used to discriminate between the target and related non-target molecule and a related, but distinct non-target molecule. The display library or a pool thereof is contacted to the non-target molecule. Members of the sample that do not bind the non-target are collected and used in subsequent selections for binding to the target molecule or even for subsequent negative selections. The negative selection step can be prior to or after selecting library members that bind to the target molecule.

In another implementation, a screening step is used. After display library members are isolated for binding to the target molecule, each isolated library member is tested for its ability to bind to a non-target molecule (e.g., a non-target listed above). For example, a high-throughput ELISA screen can be used to obtain this data. The ELISA screen can also be used to obtain quantitative data for binding of each library member to the target. The non-target and target binding data are compared (e.g., using a computer and software) to identify library members that specifically bind to the target MHC-peptide complex.

C. Diversity

Display libraries include variation at one or more positions in the displayed polypeptide. The variation at a given position can be synthetic or natural. For some libraries, both synthetic and natural diversity are included.

1. Synthetic Diversity

Libraries can include regions of diverse nucleic acid sequence that originate from artificially synthesized sequences. Typically, these are formed from degenerate oligonucleotide populations that include a distribution of nucleotides at each given position. The inclusion of a given sequence is random with respect to the distribution. One example of a degenerate source of synthetic diversity is an oligonucleotide that includes NNN wherein N is any of the four nucleotides in equal proportion.

Synthetic diversity can also be more constrained, e.g., to limit the number of codons in a nucleic acid sequence at a given trinucleotide to a distribution that is smaller than NNN. For example, such a distribution can be constructed using less than four nucleotides at some positions of the codon. In addition, trinucleotide addition technology can be used to further constrain the distribution.

So-called “trinucleotide addition technology” is described, e.g., in Wells et al. (1985) Gene 34:315-323, U.S. Pat. Nos. 4,760,025 and 5,869,644. Oligonucleotides are synthesized on a solid phase support, one codon (i.e., trinucleotide) at a time. The support includes many functional groups for synthesis such that many oligonucleotides are synthesized in parallel. The support is first exposed to a solution containing a mixture of the set of codons for the first position. The unit is protected so additional units are not added. The solution containing the first mixture is washed away and the solid support is deprotected so a second mixture containing a set of codons for a second position can be added to the attached first unit. The process is iterated to sequentially assemble multiple codons. Trinucleotide addition technology enables the synthesis of a nucleic acid that at a given position can encode a number of amino acids. The frequency of these amino acids can be regulated by the proportion of codons in the mixture. Further the choice of amino acids at the given position is not restricted to quadrants of the codon table as is the case if mixtures of single nucleotides are added during the synthesis.

2. Natural Diversity

Libraries can include regions of diverse nucleic acid sequence that originate (or are synthesized based on) from different naturally-occurring sequences. An example of natural diversity that can be included in a display library is the sequence diversity present in immune cells (see also below). Nucleic acids are prepared from these immune cells and are manipulated into a format for polypeptide display. Another example of natural diversity is the diversity of sequences between different species of organisms. For example, diverse nucleic acid sequences can be amplified from environmental samples, such as soil, and used to construct a display library.

D. Antibody Display Libraries

In one embodiment, the display library presents a diverse pool of polypeptides, each of which includes an immunoglobulin domain, e.g., an immunoglobulin variable domain. Display libraries are particularly useful, for example for identifying human or “humanized” antibodies that recognize human antigens. Such antibodies can be used as therapeutics to treat human disorders such as cancer. Since the constant and framework regions of the antibody are human, these therapeutic antibodies may avoid being recognized and targeted as antigens. The constant regions are also optimized to recruit effector functions of the human immune system. The in vitro display selection process surmounts the inability of a normal human immune system to generate antibodies against self-antigens.

A typical antibody display library displays a polypeptide that includes a VH domain and a VL domain. An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay (1988) Ann. Rev. Immunol. 6:381-405). The display library can display the antibody as a Fab fragment (e.g., using two polypeptide chains) or a single chain Fv (e.g., using a single polypeptide chain). Other formats can also be used.

As in the case of the Fab and other formats, the displayed antibody can include a constant region as part of a light or heavy chain. In one embodiment, each chain includes one constant region, e.g., as in the case of a Fab. In other embodiments, additional constant regions are displayed.

Antibody libraries can be constructed by a number of processes (see, e.g., de Haard et al. (1999) J. Biol. Chem. 274:18218-30; Hoogenboom et al. (1998) Immunotechnology 4:1-20, and Hoogenboom et al. (2000) Immunol. Today 21:371-8). Further, elements of each process can be combined with those of other processes. The processes can be used such that variation is introduced into a single immunoglobulin domain (e.g., VH or VL) or into multiple immunoglobulin domains (e.g., VH and VL). The variation can be introduced into an immunoglobulin variable domain, e.g., in the region of one or more of CDR1, CDR2, CDR3, FR1, FR2, FR3, and FR4, referring to such regions of either and/or both of heavy and light chain variable domains. In one embodiment, variation is introduced into all three CDRs of a given variable domain. In another preferred embodiment, the variation is introduced into CDR1 and CDR2, e.g., of a heavy chain variable domain. Any combination is feasible.

In one process, antibody libraries are constructed by inserting diverse oligonucleotides that encode CDRs into the corresponding regions of the nucleic acid. The oligonucleotides can be synthesized using monomeric nucleotides or trinucleotides. For example, Knappik et al. ((2000) J. Mol. Biol. 296:57-86) describe a method for constructing CDR encoding oligonucleotides using trinucleotide synthesis and a template with engineered restriction sites for accepting the oligonucleotides.

In another process, an animal, e.g., a rodent, is immunized with the MUC1-H. The animal is optionally boosted with the antigen to further stimulate the response. Then spleen cells are isolated from the animal, and nucleic acid encoding VH and/or VL domains is amplified and cloned for expression in the display library.

In yet another process, antibody libraries are constructed from nucleic acid amplified from naive germline immunoglobulin genes. The amplified nucleic acid includes nucleic acid encoding the VH and/or VL domain. Sources of immunoglobulin-encoding nucleic acids are described below. Amplification can include PCR, e.g., with primers that anneal to the conserved constant region, or another amplification method.

Nucleic acid encoding immunoglobulin domains can be obtained from the immune cells of, e.g., a human, a primate, mouse, rabbit, camel, or rodent. In one example, the cells are selected for a particular property. B cells at various stages of maturity can be selected. In another example, the B cells are naive.

In one embodiment, fluorescent-activated cell sorting (FACS) is used to sort B cells that express surface-bound IgM, IgD, or IgG molecules. Further, B cells expressing different isotypes of IgG can be isolated. In another preferred embodiment, the B or T cell is cultured in vitro. The cells can be stimulated in vitro, e.g., by culturing with feeder cells or by adding mitogens or other modulatory reagents, such as antibodies to CD40, CD40 ligand or CD20, phorbol myristate acetate, bacterial lipopolysaccharide, concanavalin A, phytohemagglutinin or pokeweed mitogen.

In still another embodiment, the cells are isolated from a subject that has an immunological disorder, e.g., systemic lupus erythematosus (SLE), rheumatoid arthritis, vasculitis, Sjogren syndrome, systemic sclerosis, or anti-phospholipid syndrome. The subject can be a human, or an animal, e.g., an animal model for the human disease, or an animal having an analogous disorder. In yet another embodiment, the cells are isolated from a transgenic non-human animal that includes a human immunoglobulin locus.

In one preferred embodiment, the cells have activated a program of somatic hypermutation. Cells can be stimulated to undergo somatic mutagenesis of immunoglobulin genes, for example, by treatment with anti-immunoglobulin, anti-CD40, and anti-CD38 antibodies (see, e.g., Bergthorsdottir et al. (2001) J. Immunol. 166:2228). In another embodiment, the cells are naive.

The nucleic acid encoding an immunoglobulin variable domain can be isolated from a natural repertoire by the following exemplary method. First, RNA is isolated from the immune cell. Full length (i.e., capped) mRNAs are separated (e.g., by degrading uncapped RNAs with calf intestinal phosphatase). The cap is then removed with tobacco acid pyrophosphatase and reverse transcription is used to produce the cDNAs.

The reverse transcription of the first (antisense) strand can be done in any manner with any suitable primer. See, e.g., de Haard et al. (1999) J. Biol. Chem. 274:18218-30. The primer binding region can be constant among different immunoglobulins, e.g., in order to reverse transcribe different isotypes of immunoglobulin. The primer binding region can also be specific to a particular isotype of immunoglobulin. Typically, the primer is specific for a region that is 3′ to a sequence encoding at least one CDR. In another embodiment, poly-dT primers may be used (and may be preferred for the heavy-chain genes).

A synthetic sequence can be ligated to the 3′end of the reverse transcribed strand. The synthetic sequence can be used as a primer binding site for binding of the forward primer during PCR amplification after reverse transcription. The use of the synthetic sequence can obviate the need to use a pool of different forward primers to fully capture the available diversity.

The variable domain-encoding gene is then amplified, e.g., using one or more rounds. If multiple rounds are used, nested primers can be used for increased fidelity. The amplified nucleic acid is then cloned into a display library vector.

Any method for amplifying nucleic acid sequences may be used for amplification. Methods that maximize, and do not bias, diversity are preferred. A variety of techniques can be used for nucleic acid amplification. The polymerase chain reaction (PCR; U.S. Pat. Nos. 4,683,195 and 4,683,202; Saiki et al. (1985) Science 230:1350-1354) utilizes cycles of varying temperature to drive rounds of nucleic acid synthesis. Transcription-based methods utilize RNA synthesis by RNA polymerases to amplify nucleic acid (U.S. Pat. No. 6,066,457; U.S. Pat. No. 6,132,997; U.S. Pat. No. 5,716,785; Sarkar et. al. (1989) Science 244: 331-34; Stofler et al. (1988) Science 239:491). NASBA (U.S. Pat. Nos. 5,130,238; 5,409,818; and 5,554,517) utilizes cycles of transcription, reverse-transcription, and RnaseH-based degradation to amplify a DNA sample. Still other amplification methods include rolling circle amplification (RCA; U.S. Pat. Nos. 5,854,033 and 6,143,495) and strand displacement amplification (SDA; U.S. Pat. Nos. 5,455,166 and 5,624,825).

E. Secondary Screening Methods

After selecting candidate display library members that bind to a target, each candidate display library member can be further analyzed, e.g., to further characterize its binding properties for the target. Each candidate display library member can be subjected to one or more secondary screening assays. The assay can be for a binding property, a catalytic property, a physiological property (e.g., cytotoxicity, renal clearance, immunogenicity), a structural property (e.g., stability, conformation, oligomerization state) or another functional property. The same assay can be used repeatedly, but with varying conditions, e.g., to determine pH, ionic, or thermal sensitivities.

As appropriate, the assays can use the display library member directly, a recombinant polypeptide produced from the nucleic acid encoding a displayed polypeptide, or a synthetic peptide synthesized based on the sequence of a displayed peptide. Exemplary assays for binding properties include the following.

1. ELISA

Polypeptides encoded by a display library can also be screened for a binding property using an ELISA assay. For example, each polypeptide is contacted to a microtitre plate whose bottom surface has been coated with the target, e.g., a limiting amount of the target. The plate is washed with buffer to remove non-specifically bound polypeptides. Then the amount of the polypeptide bound to the plate is determined by probing the plate with an antibody that can recognize the polypeptide, e.g., a tag or constant portion of the polypeptide. The antibody is linked to an enzyme such as alkaline phosphatase, which produces a calorimetric product when appropriate substrates are provided. The polypeptide can be purified from cells or assayed in a display library format, e.g., as a fusion to a filamentous bacteriophage coat. In another version of the ELISA assay, each polypeptide of a diversity strand library is used to coat a different well of a microtitre plate. The ELISA then proceeds using a constant target molecule to query each well.

2. Homogeneous Binding Assays

The binding interaction of candidate polypeptide with a target can be analyzed using a homogenous assay, i.e., after all components of the assay are added, additional fluid manipulations are not required. For example, fluorescence resonance energy transfer (FRET) can be used as a homogenous assay (see, for example, Lakowicz et al., U.S. Pat. No. 5,631,169; Stavrianopoulos et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A binding event that is configured for monitoring by FRET can be conveniently measured through standard fluorometric detection means well known in the art (e.g., using a fluorimeter). By titrating the amount of the first or second binding molecule, a binding curve can be generated to estimate the equilibrium binding constant.

Another example of a homogenous assay is Alpha Screen (Packard Bioscience, Meriden, Conn., USA). Alpha Screen uses two labeled beads. One bead generates singlet oxygen when excited by a laser. The other bead generates a light signal when singlet oxygen diffuses from the first bead and collides with it. The signal is only generated when the two beads are in proximity. One bead can be attached to the display library member, the other to the target. Signals are measured to determine the extent of binding.

The homogenous assays can be performed while the candidate polypeptide is attached to the display library vehicle, e.g., a bacteriophage.

3. Surface Plasmon Resonance (SPR)

The binding interaction of a molecule isolated from a display library and a target can be analyzed using SPR. SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons, Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (Kd), and kinetic parameters, including Kₒₙ and K_{off}, for the binding of a biomolecule to a target. Such data can be used to compare different biomolecules. For example, proteins encoded by nucleic acid selected from a library of diversity strands can be compared to identify individuals that have high affinity for the target or that have a slow K_{off}. This information can also be used to develop structure-activity relationships (SAR). For example, the kinetic and equilibrium binding parameters of matured versions of a parent protein can be compared to the parameters of the parent protein. Variant amino acids at given positions can be identified that correlate with particular binding parameters, e.g., high affinity and slow K_{off}. This information can be combined with structural modeling (e.g., using homology modeling, energy minimization, or structure determination by crystallography or NMR). As a result, an understanding of the physical interaction between the protein and its target can be formulated and used to guide other design processes.

4. Protein Arrays

Polypeptides identified from the display library can be immobilized on a solid support, for example, on a bead or an array. For a protein array, each of the polypeptides is immobilized at a unique address on a support. Typically, the address is a two-dimensional address. Protein arrays are described below (see, e.g., “Diagnostics”).

5. Cellular Assays

A library of candidate polypeptides (e.g., previously identified by a display library or otherwise) can be screened by transforming the library into a host cell. For example, the library can include vector nucleic acid sequences that include segments that encode the polypeptides and that direct expression, e.g., such that the polypeptides are produced within the cell, secreted from the cell, or attached to the cell surface. The cells can be screened for polypeptides that bind to the MUC1-H, e.g., as detected by a change in a cellular phenotype or a cell-mediated activity. For example, in the case of an antibody that binds to the MUC1-H, the activity may be cell or complement-mediated cytotoxicity.

In another embodiment, the library of cells is in the form of a cellular array. The cellular array can likewise be screened for any appropriate detectable activity.

F. Ligand Production

Standard recombinant nucleic acid methods can be used to express a polypeptide ligand that binds to MUC1-H. Generally, a nucleic acid sequence encoding the polypeptide ligand is cloned into a nucleic acid expression vector. Of course, if the protein includes multiple polypeptide chains, each chain must be cloned into an expression vector, e.g., the same or different vectors, that are expressed in the same or different cells. If the protein is sufficiently small, i.e., the protein is a peptide of less than 50 amino acids, the protein can be synthesized using automated organic synthetic methods. Methods for producing antibodies are also provided below.

The expression vector for expressing the polypeptide ligand can include, in addition to the segment encoding the polypeptide ligand or fragment thereof, regulatory sequences, including for example, a promoter, operably linked to the nucleic acid(s) of interest. Large numbers of suitable vectors and promoters are known to those of skill in the art and are commercially available for generating the recombinant constructs of the present invention. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXTI, pSG (Stratagene) pSVK3, pBPV, pMSG, and pSVL (Pharmacia). One preferred class of preferred libraries is the display library, which is described below.

Methods well known to those skilled in the art can be used to construct vectors containing a polynucleotide of the invention and appropriate transcriptional/translational control signals. These methods include in vitro recombinant DNA techniques, synthetic techniques and in vivo recombination/genetic recombination. See, for example, the techniques described in Sambrook & Russell, Molecular Cloning: A Laboratory Manual, 3^{rd} Edition, Cold Spring Harbor Laboratory, N.Y. (2001) and Ausubel et al., Current Protocols in Molecular Biology (Greene Publishing Associates and Wiley Interscience, N.Y. (1989). Promoter regions can be selected from any desired gene using CAT (chloramphenicol transferase) vectors or other vectors with selectable markers. Two appropriate vectors are pKK232-8 and pCM7. Particular named bacterial promoters include lac, lacZ, T3, T7, gpt, lambda P, and trc. Eukaryotic promoters include CMV immediate early, HSV thymidine kinase, early and late SV40, LTRs from retrovirus, mouse metallothionein-I, and various art-known tissue specific promoters.

Generally, recombinant expression vectors will include origins of replication and selectable markers permitting transformation of the host cell, e.g., the ampicillin resistance gene of E. coli and S. cerevisiae auxotrophic markers (such as URA3, LEU2, HIS3, and TRPl genes), and a promoter derived from a highly expressed gene to direct transcription of a downstream structural sequence. Such promoters can be derived from operons encoding glycolytic enzymes such as 3-phosphoglycerate kinase (PGK), a-factor, acid phosphatase, or heat shock proteins, among others. The polynucleotide of the invention is assembled in appropriate phase with translation initiation and termination sequences, and preferably, a leader sequence capable of directing secretion of translated protein into the periplasmic space or extracellular medium. Optionally, a nucleic acid of the invention can encode a fusion protein including an N-terminal identification peptide imparting desired characteristics, e.g., stabilization or simplified purification of expressed recombinant product. Useful expression-vectors for bacteria are constructed by inserting a polynucleotide of the invention together with suitable translation initiation and termination signals, optionally in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but nonlimiting example, useful expression vectors for bacteria can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and pGEM1 (Promega, Madison, Wis., USA).

The present invention further provides host cells containing the vectors of the present invention, wherein the nucleic acid has been introduced into the host cell using known transformation, transfection or infection methods. For example, the host cells can include members of a library constructed from the diversity strand. The host cell can be a eukaryotic host cell, such as a mammalian cell, a lower eukaryotic host cell, such as a yeast cell, or the host cell can be a prokaryotic cell, such as a bacterial cell. Introduction of the recombinant construct into the host cell can be effected, for example, by calcium phosphate transfection, DEAE, dextran mediated transfection, or electroporation (Davis, L. et al., Basic Methods in Molecular Biology (1986)).

Any host/vector system can be used to identify one or more of the target elements of the present invention. These include, but are not limited to, eukaryotic hosts such as HeLa cells, CV-1 cell, COS cells, and Sf9 cells, as well as prokaryotic host such as E. coli and B. subtilis. The most preferred cells are those which do not normally express the particular reporter polypeptide or protein or which expresses the reporter polypeptide or protein at low natural level.

The host of the present invention may also be a yeast or other fungi. In yeast, a number of vectors containing constitutive or inducible promoters may be used. For a review see, Current Protocols in Molecular Biology, Vol. 2, Ed. Ausubel et al., Greene Publish. Assoc. & Wiley Interscience, Ch. 13 (1988); Grant et al. (1987) “Expression and Secretion Vectors for Yeast”, Methods Enzymol. 153:516-544; Glover, DNA Clonin, Vol. II, IRL Press, Wash., D.C., Ch. 3 (1986); Bitter, Heterologous Gene Expression in Yeast, Methods Enzymol. 152:673-684 (1987); and The Molecular Biology of the Yeast Saccharomyces, Eds. Strathern et al., Cold Spring Harbor Press, Vols. I and II (1982).

The host of the invention may also be a prokaryotic cell such as E. coli, other enterobacteriaceae such as Serratia marescans, bacilli, various pseudomonads, or other prokaryotes which can be transformed, transfected, and/or infected.

The present invention further provides host cells genetically engineered to contain the polynucleotides of the invention. For example, such host cells may contain nucleic acids of the invention introduced into the host cell using known transformation, transfection or infection methods. The present invention still further provides host cells genetically engineered to express the polynucleotides of the invention, wherein such polynucleotides are in operative association with a regulatory sequence heterologous to the host cell which drives expression of the polynucleotides in the cell.

The host cell can be a higher eukaryotic host cell, such as a mammalian cell, a lower eukaryotic host cell, such as a yeast cell, or the host cell can be a prokaryotic cell, such as a bacterial cell.

Introduction of the recombinant construct into the host cell can be effected by calcium phosphate transfection, DEAE, dextran mediated transfection, or electroporation (Davis, L. et al., (1986) Basic Methods in Molecular Biology). The host cells containing one of polynucleotides of the invention, can be used in a conventional manner to produce the gene product encoded by the isolated fragment (in the case of an ORF).

Any host/vector system can be used to express one or more of the diversity strands of the present invention. These include, but are not limited to, eukaryotic hosts such as HeLa cells, CV-1 cell, COS cells, and Sf9 cells, as well as prokaryotic host such as E. coli and B. subtilis. The most preferred cells are those which do not normally express the particular polypeptide or protein or which expresses the polypeptide or protein at low natural level. Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook et al., in Molecular Cloning: A Laboratory Manual, Second Edition, Cold Spring Harbor, N.Y. (1989), the disclosure of which is incorporated herein by reference in its entirety.

Various mammalian cell culture systems can also be employed to express recombinant protein.

Examples of mammalian expression systems include the COS-7 lines of monkey kidney fibroblasts, described by Gluzman (1981) Cell 23:175 (1981), and other cell lines capable of expressing a compatible vector, for example, the C127, 3T3, CHO, HeLa and BHK cell lines. Mammalian expression vectors will comprise an origin of replication, a suitable promoter and also any necessary ribosome-binding sites, polyadenylation site, splice donor and acceptor sites, transcriptional termination sequences, and 5′ flanking nontranscribed sequences.

DNA sequences derived from the SV40 viral genome, for example, SV40 origin, early promoter, enhancer, splice, and polyadenylation sites may be used to provide the required nontranscribed genetic elements. Recombinant polypeptides and proteins produced in bacterial culture are usually isolated by initial extraction from cell pellets, followed by one or more salting-out, aqueous ion exchange or size exclusion chromatography steps. In some embodiments, the template nucleic acid also encodes a polypeptide tag, e.g., penta- or hexa-histidine. The recombinant polypeptides encoded by a library of diversity strands can then be purified using affinity chromatography.

Microbial cells employed in expression of proteins can be disrupted by any convenient method, including freeze-thaw cycling, sonication, mechanical disruption, or use of cell lysing agents. A number of types of cells may act as suitable host cells for expression of the protein. Scopes ((1994) Protein Purification: Principles and Practice, Springer-Verlag, New York) provides a number of general methods for purifying recombinant (and non-recombinant) proteins. The method include, e.g., ion-exchange chromatography, size-exclusion chromatography, affinity chromatography, selective precipitation, dialysis, and hydrophobic interaction chromatography. These methods can be adapted for devising a purification strategy for the anti-MUC1-H polypeptide ligand.

Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods. In another embodiment of the present invention, cells and tissues may be engineered to express an endogenous gene comprising the polynucleotides of the invention under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods.

Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting, including polyadenylation signals. mRNA stability elements, splice sites, leader sequences for enhancing or modifying transport or secretion properties of the protein, or other sequences which alter or improve the function or stability of protein or RNA molecules.

1. Antibody Production

Some antibodies, e.g., Fabs, can be produced in bacterial cells, e.g., E. coli cells. For example, if the Fab is encoded by sequences in a phage display vector that includes a suppressible stop codon between the display entity and a bacteriophage protein (or fragment thereof), the vector nucleic acid can be shuffled into a bacterial cell that cannot suppress a stop codon. In this case, the Fab is not fused to the gene III protein and is secreted into the media.

Antibodies can also be produced in eukaryotic cells. In one embodiment, the antibodies (e.g., scFv's) are expressed in a yeast cell such as Pichia (see, e.g., Powers et al. (2001) J. Immunol. Methods. 251:123-35), Hanseula, or Saccharomyces.

In one preferred embodiment, antibodies are produced in mammalian cells. Preferred mammalian host cells for expressing the clone antibodies or antigen-binding fragments thereof include Chinese Hamster Ovary (CHO cells) (including dhfr-CHO cells, described in Urlaub and Chasin ((1980) Proc. Natl. Acad. Sci. USA 77:4216-4220), used with a DHFR selectable marker, e.g., as described in Kaufman and Sharp ((1982) Mol. Biol. 159:601-621), lymphocytic cell lines, e.g., NSO myeloma cells and SP2 cells, COS cells, and a cell from a transgenic animal, e.g., a transgenic mammal. For example, the cell is a mammary epithelial cell.

In addition to the nucleic acid sequence encoding the diversified immunoglobulin domain, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

In an exemplary system for recombinant expression of an antibody, or antigen-binding portion thereof, of the invention, a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. For example, some antibodies can be isolated by affinity chromatography with a Protein A or Protein G.

For antibodies that include an Fc domain, the antibody production system preferably synthesizes antibodies in which the Fc region is glycosylated. For example, the Fc domain of IgG molecules is glycosylated at asparagine 297 in the CH2 domain. This asparagine is the site for modification with biantennary-type oligosaccharides. It has been demonstrated that this glycosylation is required for effector functions mediated by Fcγ receptors and complement C1q (Burton and Woof (1992) Adv. Immunol. 51:1-84; Jefferis et al. (1998) Immunol. Rev. 163:59-76). In a preferred embodiment, the Fc domain is produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly.

G. Pharmaceutical Compositions

In another aspect, the present invention provides compositions, e.g., pharmaceutically acceptable compositions, which include an anti-MUC1-H ligand, e.g., an antibody molecule, other polypeptide or peptide identified as binding to MUC1-H, or described herein, formulated together with a pharmaceutically acceptable carrier. As used herein, the term “pharmaceutical compositions” encompasses labeled ligands for in vivo imaging as well as therapeutic compositions.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., polypeptide ligand may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M. et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for administration of humans with antibodies. The preferred mode of administration is parenteral (e.g., intravenous, subcutaneous, intraperitoneal, intramuscular). In a preferred embodiment, the anti-MUC1-H ligand is administered by intravenous infusion or injection. In another preferred embodiment, the anti-MUC1-H ligand is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions typically must be sterile and stable under the conditions of manufacture and storage. A pharmaceutical composition can also be tested to insure it meets regulatory and industry standards for administration. For example, endotoxin levels in the preparation can be tested using the Limulus amebocyte lysate assay (e.g., using the kit from Bio Whittaker, e.g., lot # 7L3790 with sensitivity 0.125 EU/mL) according to the USP 24/NF 19 methods. Sterility of pharmaceutical compositions can be determined using thioglycollate medium according to the USP 24/NF 19 methods. For example, the preparation is used to inoculate the thioglycollate medium and incubated at 35° C. for 14 or more days. The medium is inspected periodically to detect growth of a microorganism.

The composition can be formulated as a solution, microemulsion, dispersion, liposome, or other ordered structure suitable to high drug concentration. Sterile injectable solutions can be prepared by incorporating the active compound (i.e., the ligand) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The anti-MUC1-H polypeptide ligands of the present invention can be administered by a variety of methods known in the art, although for many applications, the preferred route/mode of administration is intravenous injection or infusion. For example, for therapeutic applications, the anti-MUC1-H ligand can be administered by intravenous infusion at a rate of less than 30, 20, 10, 5, or 1 mg/min to reach a dose of about 1 to 100 mg/m² or 7 to 25 mg/m². The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

In certain embodiments, the ligand may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Pharmaceutical compositions can be administered with medical devices known in the art. For example, in a preferred embodiment, a pharmaceutical composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules are also known.

In certain embodiments, the compounds of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an antibody of the invention is 0.1-20 mg/kg, more preferably 1-10 mg/kg. The anti-MUC1-H antibody can be administered by intravenous infusion at a rate of less than 30, 20, 10, 5, or 1 mg/min to reach a dose of about 1 to 100 mg/m² or about 5 to 30 mg/m². For ligands smaller in molecular weight than an antibody, appropriate amounts can be proportionally less. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective amount” or a “prophylactically effective amount” of an anti-MUC1-H ligand of the invention. A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the polypeptide ligand to elicit a desired response in the individual. A therapeutically effective amount is also one in which any toxic or detrimental effects of the composition is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the invention are kits comprising the polypeptide ligand that binds to MUC1-H and instructions for use, e.g., treatment, prophylactic, or diagnostic use. In one embodiment, the instructions for diagnostic applications include the use of the anti-MUC1-H ligand (e.g., antibody or antigen-binding fragment thereof, or other polypeptide or peptide) to detect MUC1-H, in vitro, e.g., in a sample, e.g., a biopsy or cells from a patient having a cancer or neoplastic disorder, or in vivo. In another embodiment, the instructions for therapeutic applications include suggested dosages and/or modes of administration in a patient with a cancer or neoplastic disorder. The kit can further contain a least one additional reagent, such as a diagnostic or therapeutic agent, e.g., a diagnostic or therapeutic agent as described herein, and/or one or more additional anti-MUC1-H ligands, formulated as appropriate, in one or more separate pharmaceutical preparations.

H. Treatments

Polypeptide ligands that bind to MUC1-H and identified by the methodS described herein and/or detailed herein have therapeutic and prophylactic utilities. For example, these ligands can be administered to cells in culture, e.g., in vitro or ex vivo, or in a subject, e.g., in vivo, to treat, prevent, and/or diagnose a variety of disorders, such as cancers.

As used herein, the term “treat” or “treatment” is defined as the application or administration of an anti-MUC1-H antibody, alone or in combination with, a second agent to a subject, e.g., a patient, or application or administration of the agent to an isolated tissue or cell, e.g., cell line, from a subject, e.g., a patient, who has a disorder (e.g., a disorder as described herein), a symptom of a disorder or a predisposition toward a disorder, with the purpose to cure, heal, alleviate, relieve, alter, remedy, ameliorate, improve or affect the disorder, the symptoms of the disorder or the predisposition toward the disorder. Treating a cell refers to the inhibition, ablation, killing of a cell in vitro or in vivo, or otherwise reducing capacity of a cell, e.g., an aberrant cell, to mediate a disorder, e.g., a disorder as described herein (e.g., a cancerous disorder). In one embodiment, “treating a cell” refers to a reduction in the activity and/or proliferation of a cell, e.g., a hyperproliferative cell. Such reduction does not necessarily indicate a total elimination of the cell, but a reduction, e.g., a statistically significant reduction, in the activity or the number of the cell.

As used herein, an amount of an anti-MUC1-H ligand effective to treat a disorder, or a “therapeutically effective amount” refers to an amount of the ligand which is effective, upon single or multiple dose administration to a subject, in treating a cell, e.g., a cancer cell (e.g., a MUC1-H-expressing cancer cell), or in prolonging curing, alleviating, relieving or improving a subject with a disorder as described herein beyond that expected in the absence of such treatment. As used herein, “inhibiting the growth” of the neoplasm refers to slowing, interrupting, arresting or stopping its growth and metastases and does not necessarily indicate a total elimination of the neoplastic growth.

As used herein, an amount of an anti-MUC1-H ligand effective to prevent a disorder, or a “prophylactically effective amount” of the ligand refers to an amount of an anti-MUC1-H ligand, e.g., an anti-MUC1-H antibody described herein, which is effective, upon single- or multiple-dose administration to the subject, in preventing or delaying the occurrence of the onset or recurrence of a disorder, e.g., a cancer.

The terms “induce”, “inhibit”, “potentiate”, “elevate”, “increase”, “decrease” or the like, e.g., which denote quantitative differences between two states, refer to a difference, e.g., a statistically significant difference, between the two states. For example, “an amount effective to inhibit the proliferation of the MUC1-H-expressing hyperproliferative cells” means that the rate of growth of the cells will be different, e.g., statistically significantly different, from the untreated cells.

As used herein, the term “subject” is intended to include human and non-human animals. Preferred human animals include a human patient having a disorder characterized by abnormal cell proliferation or cell differentiation. The term “non-human animals” of the invention includes all vertebrates, e.g., non-mammals (such as chickens, amphibians, reptiles) and mammals, such as non-human primates, sheep, dog, cow, pig, etc.

In one embodiment, the subject is a human subject. Alternatively, the subject can be a mammal expressing a MUC1-H-like antigen with which an antibody of the invention cross-reacts. A polypeptide ligand of the invention can be administered to a human subject for therapeutic purposes (discussed further below). Moreover, an anti-MUC1-H ligand can be administered to a non-human mammal expressing the MUC1-H-like antigen to which the ligand binds (e.g., a primate, pig or mouse) for veterinary purposes or as an animal model of human disease. Regarding the latter, such animal models may be useful for evaluating the therapeutic efficacy of the ligand (e.g., testing of dosages and time courses of administration).

In one embodiment, the invention provides a method of treating (e.g., ablating or killing) a cell (e.g., a non-cancerous cell, e.g., a normal, benign or hyperplastic cell, or a cancerous cell, e.g., a malignant cell, e.g., cell found in a solid tumor, a soft tissue tumor, or a metastatic lesion (e.g., a cell found in renal, urothelial, colonic, rectal, pulmonary, breast or hepatic, cancers and/or metastasis)). Methods of the invention include the steps of contacting the cell with an anti-MUC1-H ligand, e.g., an anti-MUC1-H antibody described herein, in an amount sufficient to treat, e.g., ablate or kill, the cell.

The subject method can be used on cells in culture, e.g., in vitro or ex vivo. For example, cancerous or metastatic cells (e.g., renal, urothelial, colon, rectal, lung, breast, ovarian, prostatic, or liver cancerous or metastatic cells) can be cultured in vitro in culture medium and the contacting step can be effected by adding the anti-MUC1-H ligand to the culture medium. The method can be performed on cells (e.g., cancerous or metastatic cells) present in a subject, as part of an in vivo (e.g., therapeutic or prophylactic) protocol. For in vivo embodiments, the contacting step is effected in a subject and includes administering the anti-MUC1-H ligand to the subject under conditions effective to permit both binding of the ligand to the cell and the treating, e.g., the killing or ablating of the cell.

Most MUC-H epitopes are present on the residual non-shed epitope of MUC1, which remains on the cell surface upon shedding, and is the extra-cellular N-terminal region of the transmembranous part of MUC1, located just before the membranous part of MUC1. This part of the protein is predicted to have no O-glycosylation sites and one putative N-glycosylation site. Because of the detachment of the shed MUC1, neo-epitopes of the transmembranous part of MUC1 are revealed that are not present on the membrane bound MUC1 molecule, and are not present on other splicing variants of MUC1. In some instances MUC1-H epitopes are present on the complex formed by the components of the clipped form of MUC1; such epitopes are not present on shed MUC1, thus are per definition MUC1-H epitopes. Because of the non-apical expression of MUC in tumors as compared with the apical expression in normal cells, MUC1-H can be construed to be a tumor-associated antigen. Antibodies and peptides against MUC1-H can therefore show tumor-specificity and MUC1-H will not be shed into the serum where it can interfere with therapeutic antibodies.

The method can be used to treat a cancer. As used herein, the terms “cancer”, “hyperproliferative”, “malignant”, and “neoplastic” are used interchangeably, and refer to those cells an abnormal state or condition characterized by rapid proliferation or neoplasm. The terms include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth.

The common medical meaning of the term “neoplasia” refers to “new cell growth” that results as a loss of responsiveness to normal growth controls, e.g., to neoplastic cell growth. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be benign, premalignant or malignant.

Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), and genitourinary tract (e.g., renal, urothelial cells), pharynx, prostate, ovary as well as adenocarcinomas which include malignancies such as most colon cancers, rectal cancer, renal-cell carcinoma, liver cancer, non-small cell carcinoma of the lung, cancer of the small intestine and so forth. Metastatic lesions of the aforementioned cancers can also be treated or prevented using the methods and compositions of the invention.

The subject method can be useful in treating malignancies of the various organ systems, such as those affecting lung, breast, lymphoid, gastrointestinal (e.g., colon), and genitourinary tract, prostate, ovary, pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus. Exemplary solid tumors that can be treated include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The term “carcinoma” is recognized by those skilled in the art and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is recognized by those skilled in the art and refers to malignant tumors of mesenchymal derivation.

The subject method can also be used to inhibit the proliferation of hyperplastic/neoplastic cells of hematopoietic origin, e.g., arising from myeloid, lymphoid or erythroid lineages, or precursor cells thereof. For instance, the present invention contemplates the treatment of various myeloid disorders including, but not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97). Lymphoid malignancies which may be treated by the subject method include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas contemplated by the treatment method of the present invention include, but are not limited to, non-Hodgkin's lymphoma and variants thereof, peripheral T-cell lymphomas, adult T-cell leukemia/lymphoma (ATL), cutaneous T-cell lymphoma (CTCL), large granular lymphocytic leukemia (LGF) and Hodgkin's disease.

Methods of administering anti-MUC1-H ligands are described in “Pharmaceutical Compositions”. Suitable dosages of the molecules used will depend on the age and weight of the subject and the particular drug used. The ligands can be used as competitive agents to inhibit, reduce an undesirable interaction, e.g., between a natural or pathological agent and the MUC1-H.

In one embodiment, the anti-MUC1-H ligands are used to kill or ablate cancerous cells and normal, benign hyperplastic, and cancerous cells in vivo. The ligands can be used by themselves or conjugated to an agent, e.g., a cytotoxic drug, radioisotope. This method includes administering the ligand alone or attached to a cytotoxic drug, to a subject requiring such treatment.

The terms “cytotoxic agent” and “cytostatic agent” and “anti-tumor agent” are used interchangeably herein and refer to agents that have the property of inhibiting the growth or proliferation (e.g., a cytostatic agent), or inducing the killing, of hyperproliferative cells, e.g., an aberrant cancer cell. In cancer therapeutic embodiment, the term “cytotoxic agent” is used interchangeably with the terms “anti-cancer” or “anti-tumor” to mean an agent, which inhibits the development or progression of a neoplasm, particularly a solid tumor, a soft tissue tumor, or a metastatic lesion.

Nonlimiting examples of anti-cancer agents include, e.g., antimicrotubule agents, topoisomerase inhibitors, antimetabolites, mitotic inhibitors, alkylating agents, intercalating agents, agents capable of interfering with a signal transduction pathway, agents that promote apoptosis, radiation, and antibodies against other tumor-associated antigens (including naked antibodies, immunotoxins and radioconjugates). Examples of the particular classes of anti-cancer agents are provided in detail as follows: antitubulin/antimicrotubule, e.g., paclitaxel, vincristine, vinblastine, vindesine, vinorelbin, taxotere; topoisomerase I inhibitors, e.g., topotecan, camptothecin, doxorubicin, etoposide, mitoxantrone, daunorubicin, idarubicin, teniposide, amsacrine, epirubicin, merbarone, piroxantrone hydrochloride; antimetabolites, e.g., 5-fluorouracil (5-FU), methotrexate, 6-mercaptopurine, 6-thioguanine, fludarabine phosphate, cytarabine/Ara-C, trimetrexate, gemcitabine, acivicin, alanosine, pyrazofurin, N-Phosphoracetyl-L-Asparate=PALA, pentostatin, 5-azacitidine, 5-Aza 2′-deoxycytidine, ara-A, cladribine, 5-fluorouridine, FUDR, tiazofurin, N-[5-[N-(3,4-dihydro-2-methyl-4-oxoquinazolin-6-ylmethyl)-N-methylamino]-2-thenoyl]-L-glutamic acid; alkylating agents, e.g., cisplatin, carboplatin, mitomycin C, BCNU=Carmustine, melphalan, thiotepa, busulfan, chlorambucil, plicamycin, dacarbazine, ifosfamide phosphate, cyclophosphamide, nitrogen mustard, uracil mustard, pipobroman, 4-ipomeanol; agents acting via other mechanisms of action, e.g., dihydrolenperone, spiromustine, and desipeptide; biological response modifiers, e.g., to enhance anti-tumor responses, such as interferon; apoptotic agents, such as actinomycin D; and anti-hormones, for example anti-estrogens such as tamoxifen or, for example antiandrogens such as 4′-cyano-3-(4-fluorophenylsulphonyl)-2-hydroxy-2-methyl-3′-(trifluoromethyl)propionanilide.

Since the anti-MUC1-H ligands recognize normal epithelial cells, any such cells to which the ligands bind are destroyed. Alternatively, the ligands bind to cells in the vicinity of the cancerous cells and kill them, thus indirectly attacking the cancerous cells which may rely on surrounding cells for nutrients, growth signals and so forth. Thus, the anti-MUC1-H ligands (e.g., modified with a cytotoxin) can selectively kill or ablate cells in cancerous tissue (including the cancerous cells themselves).

The ligands may be used to deliver a variety of cytotoxic drugs including therapeutic drugs, a compound emitting radiation, molecules of plants, fungal, or bacterial origin, biological proteins, and mixtures thereof. The cytotoxic drugs can be intracellularly acting cytotoxic drugs, such as short-range radiation emitters, including, for example, short-range, high-energy α-emitters, as described herein.

Enzymatically active toxins and fragments thereof are exemplified by diphtheria toxin A fragment, nonbinding active fragments of diphtheria toxin, exotoxin A (from Pseudomonas aeruginosa), ricin A chain, abrin A chain, modeccin A chain, α-sacrin, certain Aleurites fordii proteins, certain Dianthin proteins, Phytolacca americana proteins (PAP, PAPII and PAP-S), Morodica charantia inhibitor, curcin, crotin, Saponaria officinalis inhibitor, gelonin, mitogillin, restrictocin, phenomycin, and enomycin. Procedures for preparing enzymatically active polypeptides of the immunotoxins are described in WO 84/03508 and WO 85/03508, which are incorporated herein by reference in their entirety, and in the appended Examples below. Examples of cytotoxic moieties that can be conjugated to the antibodies include adriamycin, chlorambucil, daunomycin, methotrexate, neocarzinostatin, and platinum.

In the case of polypeptide toxins, recombinant nucleic acid techniques can be used to construct a nucleic acid that encodes the ligand (or a polypeptide component thereof) and the cytotoxin (or a polypeptide component thereof) as translational fusions. The recombinant nucleic acid is then expressed, e.g., in cells and the encoded fusion polypeptide isolated.

Procedures for conjugating polypeptide ligands (e.g., antibodies) with the cytotoxic agents have been previously described. Procedures for conjugating chlorambucil with antibodies are described by Flechner (1973) European Journal of Cancer 9:741-745; Ghose et al. (1972) British Medical Journal 3:495-499; and Szekerke et al. (1972) Neoplasma, 19:211-215, which are hereby incorporated by reference in their entirety. Procedures for conjugating daunomycin and adriamycin to antibodies are described by Hurwitz, E. et al. (1975) Cancer Research, 35:1175-1181 and Arnon et al. (1982) Cancer Surveys, 1:429-449, which are hereby incorporated by reference in their entirety. Procedures for preparing antibody-ricin conjugates are described in U.S. Pat. No. 4,414,148 and by Osawa, T. et al. (1982) Cancer Surveys 1:373-388 and the references cited therein, which are hereby incorporated by reference in their entirety. Coupling procedures as also described in published European patent application EP 86309516.2, which is hereby incorporated by reference in its entirety.

To kill or ablate normal, benign hyperplastic, or cancerous cells, a first polypeptide ligand is conjugated with a prodrug which is activated only when in close proximity with a prodrug activator. The prodrug activator is conjugated with a second polypeptide ligand, preferably one which binds to a non-competing site on the target molecule. Whether two polypeptide ligands bind to competing or non-competing binding sites can be determined by conventional competitive binding assays. Drug-prodrug pairs suitable for use in the practice of the present invention are described in Blakely et al. (1996) Cancer Research 56:3287-3292.

Alternatively, the anti-MUC1-H ligand can be coupled to high energy radiation emitters, for example, a radioisotope, such as ¹³¹¹I, α γ-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (eds.), pp 303-316 (Academic Press 1985). Other suitable radioisotopes include α-emitters, such as ²¹²Bi, ²¹³Bi, and ²¹¹At, and β-emitters, such as ¹⁸⁶Re and ⁹⁰Y. Moreover, Lu¹¹⁷ may also be used as both an imaging and cytotoxic agent.

Radioimmunotherapy (RIT) using antibodies labeled with ¹³¹I ,⁹⁰Y, and ¹⁷⁷Lu is under intense clinical investigation. There are significant differences in the physical characteristics of these three nuclides and as a result, the choice of radionuclide is very critical in order to deliver maximum radiation dose to the tumor. The higher beta energy particles of ⁹⁰Y may be good for bulky tumors. The relatively low energy beta particles of ¹³¹I are ideal, but in vivo dehalogenation of radioiodinated molecules is a major disadvantage for internalizing antibody. In contrast, ¹⁷⁷Lu has low energy beta particle with only 0.2-0.3 mm range and delivers much lower radiation dose to bone marrow compared to ⁹⁰Y. In addition, due to longer physical half-life (compared to ⁹⁰Y), the tumor residence times are higher. As a result, higher activities (more mCi amounts) of ¹⁷⁷Lu labeled agents can be administered with comparatively less radiation dose to marrow. There have been several clinical studies investigating the use of ¹⁷⁷Lu labeled antibodies in the treatment of various cancers. (Mulligan, T. et al. (1995) Clin. Cancer Res. 1:1447-1454; Meredith, R. F. et al. (1996) J. Nucl. Med. 37:1491-1496; Alvarez, R. D. et al. (1997) Gynecologic Oncology 65:94-101).

The anti-MUC1-H ligands can be used directly in vivo to eliminate antigen-expressing cells via natural complement-dependent cytotoxicity (CDC) or antibody-dependent cellular cytotoxicity (ADCC). The polypeptide ligands of the invention, can include complement binding effector domain, such as the Fe portions from IgG1, -2, or -3 or corresponding portions of IgM which bind complement. In one embodiment, a population of target cells is ex vivo treated with a binding agent of the invention and appropriate effector cells. The treatment can be supplemented by the addition of complement or serum containing complement. Further, phagocytosis of target cells coated with a polypeptide ligand of the invention can be improved by binding of complement proteins. In another embodiment target, cells coated with the polypeptide ligand which includes a complement binding effector domain are lysed by complement.

Also encompassed by the present invention is a method of killing or ablating which involves using the anti-MUC1-H ligand for prophylaxis. For example, these materials can be used to prevent or delay development or progression of cancers.

Use of the therapeutic methods of the present invention to treat cancers has a number of benefits. Since the polypeptide ligands specifically recognize MUC1-H, other tissue is spared and high levels of the agent are delivered directly to the site where therapy is required. Treatment in accordance with the present invention can be effectively monitored with clinical parameters. Alternatively, these parameters can be used to indicate when such treatment should be employed.

Anti-MUC1-H ligands of the invention can be administered in combination with one or more of the existing modalities for treating cancers, including, but not limited to: surgery, radiation therapy, and chemotherapy.

I. Diagnostic Uses

Polypeptide ligands that bind to MUC1-H and identified by the methodS described herein and/or detailed herein have in vitro and in vivo diagnostic, therapeutic and prophylactic utilities.

In one aspect, the present invention provides a diagnostic method for detecting the presence of a MUC1-H, in vitro (e.g., a biological sample, such as tissue, biopsy, e.g., a cancerous tissue) or in vivo (e.g., in vivo imaging in a subject).

The method includes: (i) contacting a sample with anti-MUC1-H ligand; and (ii) detecting formation of a complex between the anti-MUC1-H ligand and the sample. The method can also include contacting a reference sample (e.g., a control sample) with the ligand, and determining the extent of formation of the complex between the ligand and the sample relative to the same for the reference sample. A change, e.g., a statistically significant change, in the formation of the complex in the sample or subject relative to the control sample or subject can be indicative of the presence of MUC1-H in the sample.

Another method includes: (i) administering the anti-MUC1-H ligand to a subject; and (iii) detecting formation of a complex between the anti-MUC1-H ligand, and the subject. The detecting can include determining location or time of formation of the complex.

The anti-MUC1-H ligand can be directly or indirectly labeled with a detectable substance to facilitate detection of the bound or unbound antibody. Suitable detectable substances include various enzymes, prosthetic groups, fluorescent materials, luminescent materials and radioactive materials.

Complex formation between the anti-MUC1-H ligand and MUC1-H can be detected by measuring or visualizing either the ligand bound to the MUC1-H or unbound ligand. Conventional detection assays can be used, e.g., an enzyme-linked immunosorbent assays (ELISA), a radioimmunoassay (RIA) or tissue immunohistochemistry. Further to labeling the anti-MUC1-H ligand, the presence of MUC1-H can be assayed in a sample by a competition immunoassay utilizing standards labeled with a detectable substance and an unlabeled anti-MUC1-H ligand. In one example of this assay, the biological sample, the labeled standards and the MUC1-H binding agent are combined and the amount of labeled standard bound to the unlabeled ligand is determined. The amount of MUC1-H in the sample is inversely proportional to the amount of labeled standard bound to the MUC1-H binding agent.

Fluorophore and chromophore labeled polypeptide ligands can be prepared. Since antibodies and other proteins absorb light having wavelengths up to about 310 nm, the fluorescent moieties should be selected to have substantial absorption at wavelengths above 310 nm and preferably above 400 nm. A variety of suitable fluorescers and chromophores are described by Stryer (1968) Science 162:526 and Brand, L. et al. (1972) Annual Review of Biochemistry 41:843-868. The polypeptide ligands can be labeled with fluorescent chromophore groups by conventional procedures such as those disclosed in U.S. Pat. Nos. 3,940,475, 4,289,747, and 4,376,110. One group of fluorescers having a number of the desirable properties described above is the xanthene dyes, which include the fluoresceins and rhodamines. Another group of fluorescent compounds are the naphthylamines. Once labeled with a fluorophore or chromophore, the polypeptide ligand can be used to detect the presence or localization of the MUC1-H in a sample, e.g., using fluorescent microscopy (such as confocal or deconvolution microscopy).

1. Histological Analysis

Immunohistochemistry can be performed using the polypeptide ligands described herein. For example, in the case of an antibody, the antibody can synthesized with a label (such as a purification or epitope tag), or can be detectably labeled, e.g., by conjugating a label or label-binding group. For example, a chelator can be attached to the antibody. The antibody is then contacted to a histological preparation, e.g., a fixed section of tissue that is on a microscope slide. After an incubation for binding, the preparation is washed to remove unbound antibody. The preparation is then analyzed, e.g., using microscopy, to identify if the antibody bound to the preparation.

Of course, the antibody (or other polypeptide or peptide) can be unlabeled at the time of binding. After binding and washing, the antibody is labeled in order to render it detectable.

2. Protein Arrays

The anti-MUC1-H ligand can also be immobilized on a protein array. The protein array can be used as a diagnostic tool, e.g., to screen medical samples (such as isolated cells, blood, sera, biopsies, and the like). Of course, the protein array can also include other ligands, e.g., that bind to the MUC1-H.

Methods of producing polypeptide arrays are described, e.g., in De Wildt et al. (2000) Nature Biotech. 18:989-994; Lueking et al. (1999) Anal. Biochem. 270:103-111; Ge (2000) Nuc. Acids Res. 28:e3; MacBeath and Schreiber (2000) Science 289:1760-1763; WO 01/40803 and WO 99/51773A1. Polypeptides for the array can be spotted at high speed, e.g., using commercially available robotic apparati, e.g., from Genetic MicroSystems and Affymetrix (Santa Clara, Calif., USA) or BioRobotics (Cambridge, UK). The array substrate can be, for example, nitrocellulose, plastic, glass, e.g., surface-modified glass. The array can also include a porous matrix, e.g., acrylamide, agarose, or another polymer.

For example, the array can be an array of antibodies, e.g., as described in De Wildt, supra. Cells that produce the polypeptide ligands can be grown on a filter in an arrayed format. Polypeptide production is induced, and the expressed polypeptides are immobilized to the filter at the location of the cell.

A protein array can be contacted with a labeled target to determine the extent of binding of the target to each immobilized polypeptide from the diversity strand library. If the target is unlabeled, a sandwich method can be used, e.g., using a labeled probed, to detect binding of the unlabeled target.

Information about the extent of binding at each address of the array can be stored as a profile, e.g., in a computer database. The protein array can be produced in replicates and used to compare binding profiles, e.g., of a target and a non-target. Thus, protein arrays can be used to identify individual members of the diversity strand library that have desired binding properties with respect to one or more molecules.

3. FACS (Fluorescent Activated Cell Sorting)

The anti-MUC1-H ligand can be used to label cells, e.g., cells in a sample (e.g., a patient sample). The ligand is also attached (or attachable) to a fluorescent compound. The cells can then be sorted using fluorescent activated cell sorted (e.g., using a sorter available from Becton Dickinson Immunocytometry Systems, San Jose, Calif., USA; see also U.S. Pat. Nos. 5,627,037; 5,030,002; and 5,137,809). As cells pass through the sorter, a laser beam excites the fluorescent compound while a detector counts cells that pass through and determines whether a fluorescent compound is attached to the cell by detecting fluorescence. The amount of label bound to each cell can be quantified and analyzed to characterize the sample.

The sorter can also deflect the cell and separate cells bound by the ligand from those cells not bound by the ligand. The separated cells can be cultured and/or characterized.

4. In vivo Imaging

In still another embodiment, the invention provides a method for detecting the presence of a MUC1-H-expressing cancerous tissue in vivo. The method includes (i) administering to a subject (e.g., a patient having a cancer or neoplastic disorder) an anti-MUC1-H antibody, conjugated to a detectable marker; (ii) exposing the subject to a means for detecting said detectable marker to the MUC1-H-expressing tissues or cells. For example, the subject is imaged, e.g., by NMR or other tomographic means.

Examples of labels useful for diagnostic imaging in accordance with the present invention include radiolabels such as ¹³¹I, ¹¹¹In, ¹²³I, ^{99m}Tc, ³²P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed. The polypeptide ligand can be labeled with such reagents using known techniques. For example, see Wensel and Meares (1983) Radioimmunoimaging and Radioimmunotherapy, Elsevier, N.Y. for techniques relating to the radiolabeling of antibodies and D. Colcher et al. (1986) Methods Enzymol. 121:802-816.

A radiolabeled ligand of this invention can also be used for in vitro diagnostic tests. The specific activity of a isotopically-labeled ligand depends upon the half-life, the isotopic purity of the radioactive label, and how the label is incorporated into the antibody.

Procedures for labeling polypeptides with the radioactive isotopes (such as ¹⁴C, ³H, ³⁵S, ¹²⁵I, ³²P, ¹³¹I) are generally known. For example, tritium labeling procedures are described in U.S. Pat. No. 4,302,438. Iodinating, tritium labeling, and ³⁵S labeling procedures, e.g., as adapted for murine monoclonal antibodies, are described, e.g., by Goding, J. W. (Monoclonal Antibodies: Principles And Practice: Production And Application Of Monoclonal Antibodies In Cell Biology, Biochemistry, And Immunology 2nd ed., London, Orlando, Academic Press (1986) polypeptide. 124-126) and the references cited therein. Other procedures for iodinating polypeptides, such as antibodies, are described by Hunter and Greenwood (1962) Nature 144:945, David et al. (1974) Biochemistry 13:1014-1021, and U.S. Pat. Nos. 3,867,517 and 4,376,110. Radiolabeling elements which are useful in imaging include ¹²³I, ¹³¹I, ¹¹¹In, and ^{99m}Tc, for example. Procedures for iodinating antibodies are described by Greenwood, F. et al. (1963) Biochem. J 89:114-123; Marchalonis, J. (1969) Biochem. J. 113:299-305; and Morrison, M. et al. (1971) Immunochemistry 8:289-297. Procedures for ^{99m}Tc-labeling are described by Rhodes, B. et al. in Burchiel, S. et al. (eds.), Tumor Imaging: The Radioimmunochemical Detection of Cancer, New York: Masson 111-123 (1982) and the references cited therein. Procedures suitable for ¹¹¹In-labeling antibodies are described by Hnatowich, D. J. et al. (1983) J. Immun. Methods 65:147-157, Hnatowich, D. et al. (1984) J. Applied Radiation 35:554-557, and Buckley, R. G. et al. (1984) F.E.B.S. Lett. 166:202-204.

In the case of a radiolabeled ligand, the ligand is administered to the patient, is localized to the tumor bearing the antigen with which the ligand reacts, and is detected or “imaged” in vivo using known techniques such as radionuclear scanning using e.g., a gamma camera or emission tomography. See, e.g., A. R. Bradwell et al., “Developments in Antibody Imaging”, Monoclonal Antibodies for Cancer Detection and Therapy, R. W. Baldwin et al., (eds.), polypeptide. 65-85, Academic Press (1985). Alternatively, a positron emission transaxial tomography scanner, such as designated Pet VI located at Brookhaven National Laboratory, can be used where the radiolabel emits positrons (e.g., ¹¹C, ¹⁸F, ¹⁵O, and ¹³N).

5. MRI Contrast Agents

Magnetic Resonance Imaging (MRI) uses NMR to visualize internal features of living subject, and is useful for prognosis, diagnosis, treatment, and surgery. MRI can be used without radioactive tracer compounds for obvious benefit. Some MRI techniques are summarized in published European patent application EP-A-0 502 814. Generally, the differences related to relaxation time constants T1 and T2 of water protons in different environments is used to generate an image. However, these differences can be insufficient to provide sharp high resolution images.

The differences in these relaxation time constants can be enhanced by contrast agents. Examples of such contrast agents include a number of magnetic agents paramagnetic agents (which primarily alter T1) and ferromagnetic or superparamagnetic (which primarily alter T2 response). Chelates (e.g., EDTA, DTPA and NTA chelates) can be used to attach (and reduce toxicity) of some paramagnetic substances (e.g., Fe⁺³, Mn⁺², Gd⁺³). Other agents can be in the form of particles, e.g., less than 10 μm to about 10 nM in diameter). Particles can have ferromagnetic, antiferromagnetic or superparamagnetic properties. Particles can include, e.g., magnetite (Fe₃O₄), γ-Fe₂O₃, ferrites, and other magnetic mineral compounds of transition elements. Magnetic particles may include one or more magnetic crystals with and without nonmagnetic material. The nonmagnetic material can include synthetic or natural polymers such as sepharose, dextran, dextrin, starch and the like.

The anti-MUC1-H ligands can also be labeled with an indicating group containing of the NMR-active ¹⁹F atom, or a plurality of such atoms inasmuch as (i) substantially all of naturally abundant fluorine atoms are the ¹⁹F isotope and, thus, substantially all fluorine-containing compounds are NMR-active; (ii) many chemically active polyfluorinated compounds such as trifluoracetic anhydride are commercially available at relatively low cost, and (iii) many fluorinated compounds have been found medically acceptable for use in humans such as the perfluorinated polyethers utilized to carry oxygen as hemoglobin replacements. After permitting such time for incubation, a whole body MRI is carried out using an apparatus such as one of those described by Pykett (1982) Scientific American 246:78-88 to locate and image cancerous tissues.

Also within the scope of the invention are kits comprising the polypeptide ligand that binds to MUC1-H and instructions for diagnostic use, e.g., the use of the anti-MUC1-H ligand (e.g., antibody or antigen-binding fragment thereof, or other polypeptide or peptide) to detect MUC1-H, in vitro, e.g., in a sample, e.g., a biopsy or cells from a patient having a cancer or neoplastic disorder, or in vivo, e.g., by imaging a subject. The kit can further contain a least one additional reagent, such as a label or additional diagnostic agent. For in vivo use the ligand can be formulated as a pharmaceutical composition.

The following invention is further illustrated by the following examples, which should not be construed as further limiting. The contents of all references, pending patent applications and published patents, cited throughout this application are hereby expressly incorporated by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. An isolated polypeptide ligand which specifically binds to an epitope on MUC1 that is not present on shed MUC1 but is present on any cell-surface expressed form of MUC1.

2. An isolated polypeptide ligand which specifically binds to a cell-surface epitope on MUC1 that is not present on shed MUC1.

3. An isolated polypeptide ligand which specifically binds to an epitope on MUC1 that is not present on shed MUC1 and is not present on the MUC1-Stubble when part of the full-length non-clipped MUC1 protein.

4. A isolated polypeptide ligand which specifically binds the C-terminal 65 amino acids of the extracellular region of the MUC1 protein.

5. The isolated polypeptide ligand of claim 1, wherein said polypeptide ligand does not bind the VNTR region of the MUC1 protein.

6. The isolated polypeptide ligand of claim 1, wherein the polypeptide ligand is a peptide ligand.

7. The isolated polypeptide ligand of claim 6, wherein the polypeptide ligand is a scaffold peptide, a linear peptide, or a cyclic peptide.

8. The isolated polypeptide ligand of claim 1, wherein the polypeptide ligand is an antibody.

9. The antibody of claim 8, wherein the antibody specifically binds a polypeptide consisting of SEQ ID NO: 2.

10. The antibody of claim 8, wherein the antibody is a human antibody.

11. The antibody of claim 8, wherein the antibody is an intact immunoglobulin.

12. The antibody of claim 8, wherein the antibody is conjugated to a functional moiety.

13. The antibody of claim 12, wherein the functional moiety is a drug, a cytotoxic agent, a detectable moiety, or a solid support.

14. An isolated antibody which can compete with the antibody of claim 9 for binding to SEQ ID NO: 2.

15. An isolated antibody which can compete with the antibody of claim 9 for binding to a complex between SEQ ID NO: 2. and SEQ ID NO: 1.

16. The antibody of claim 9, wherein the antibody specifically binds a polypeptide consisting of the N-terminal 51 amino acids of SEQ ID NO: 2.

17. An isolated non-antibody polypeptide ligand which specifically binds a polypeptide consisting of SEQ ID NO: 2.

18. The isolated non-antibody polypeptide ligand of claim 17, wherein said polypeptide ligand does not bind the VNTR region of the MUC1 protein.

19. The isolated non-antibody polypeptide ligand of claim 17, wherein said ligand specifically binds a polypeptide consisting of the first 51 amino acids of SEQ ID NO: 2.

20. The isolated non-antibody polypeptide ligand of claim 17, wherein the polypeptide ligand is conjugated to a functional moiety.

21. The isolated non-antibody polypeptide ligand of claim 17, wherein the functional moiety is a drug, a cytotoxic agent, a detectable moiety, or a solid support.

22. A method of detecting MUC1-H in a sample, the method comprising:
(a) providing a sample;
(b) contacting the sample of (a) with a polypeptide ligand which specifically binds a polypeptide comprising MUC1-H under conditions which permit binding of the polypeptide ligand to MUC1-H; and
(c) detecting binding of the polypeptide ligand with MUC1-H in the sample,
wherein detection of binding indicates the presence of MUC1-H in the sample;
thereby detecting MUC1-H in the sample.

23. A method of identifying a polypeptide ligand specific for MUC1-H, comprising:
(a) providing a phage library comprising phage expressing candidate MUC1-H binding polypeptides;
(b) contacting said phage library with MUC1-H protein; and
(c) detecting binding of the MUC1-H protein to phage;
thereby identifying a polypeptide ligand specific for MUC1-H.

24. A method of killing a cell, the method comprising:
(a) providing a cell;
(b) contacting the cell of (a) with a polypeptide ligand which specifically binds a polypeptide comprising MUC1-H under conditions which permit binding of the polypeptide ligand to MUC1-H; and
thereby killing the cell.
